(19) United States
(12) Patent Application Publication
(10) Pub. No.: US 2024/0377371 A1
(43) Pub. Date: Nov. 14, 2024

(54) METHOD FOR ESTIMATING SELECTIVITY AND/OR ACTIVITY OF A CATALYST IN AN ETHYLENE OXIDE REACTOR

(71) Applicant: Scientific Design Company, Inc., Little Ferry, NJ (US)

(72) Inventors: VIKAS KUMAR CHALKA, Dubai (AE); Mansoor HUSAIN, North Brunswick, NJ (US); SOMNATH CHOWDHURY, Dubai (AE); SANDIP KUMAR LAHIRI, West Bengal (IN)

(73) Assignee: Scientific Design Company, Inc., Little Ferry, NJ (US)

(21) Appl. No.: 18/654,774

(22) Filed: May 3, 2024

Related U.S. Application Data
(60) Provisional application No. 63/463,631, filed on May 3, 2023.

(51) Int. Cl. G01N 30/86; G01N 30/72

(52) U.S. Cl. CPC G01N 30/8651; G01N 30/7206

(57) ABSTRACT

The invention relates to a method of generating a signal for adjusting a parameter of a process for ethylene oxide production comprising the steps:
Acquiring process data of a reactor producing ethylene oxide by means of sensors,
Determining a parametric coefficient from the acquired process data, and
Calculating the selectivity and/or activity of the catalyst from the parametric coefficient.

### CROSS REFERENCE TO RELATED APPLICATION

The present invention claims the benefit of U.S. Provisional Patent Application No. 63/463,631 filed May 3, 2023, the entire content and disclosure of which is incorporated herein by reference.

### FIELD OF INVENTION

This invention relates to optimizing commercial ethylene oxide (EO) production in a reactor and more particularly to a method for selectivity and/or activity of a catalyst in an ethylene oxide reactor.

### BACKGROUND OF THE INVENTION

Business environment has encountered a drastic transformation in this decade due to stiff global competition. Chemical process industries are facing a perpetual shrink in profit margin due to the rise in competition in the turbulent market, thanks to globalization. Technological innovation to improve process and energy efficiency, yield maximization, and environmental impact reduction are the only path to mitigate these issues successfully. Chemical companies across the globe are striving to find new innovative ways to reduce production cost and maximize profit. Application of artificial intelligence-based techniques to generate value from large amount of plant operating data by data mining and knowledge discovery is one of the most attractive innovative ways to explore. In search of innovative ways to generate more profit, chemical reactor attracts the focus of plant managers and researchers. In chemical plants, the reactor is the only large piece of equipment that converts raw materials into finished goods and adds real value. In this regard, reactor optimization has an incredible potential impact on overall plant profitability. ^{[1]}To optimize the commercial reactor, it is required to model complex systems of industrial chemical reactions as a first step. Industrial chemical reactions involved complicated reaction kinetics and thermodynamics. Building a credible phenomenological model for commercial reactors is considered as a very time-consuming difficult task as it demands a deep knowledge of reaction kinetics and understanding of industrial heterogeneous catalytic behavior characterized by mass diffusion, catalyst deactivation, etc. Lack of sufficient knowledge in chemical reaction kinetics actually hinders the optimization of the reactor. In most chemical plants, the reactors remain as black box, and operation engineers do not try experimentation in running plants due to safety and reliability reasons. This drives non-optimum operation of industrial reactors, which leads to loss of huge money and greatly affects the profitability of the plant. However, a small increment of catalyst selectivity and reaction yield has large potential to significantly affect the raw material consumption and plant profitability in large-scale operation.

Data is considered as new oil in current time, and certainly, artificial intelligence based data driven modeling techniques like Artificial neural network (ANN), support vector machine (SVM), genetic programing (GP) etc. are considered new IC engine (internal combustion engine) of current age. As huge reactor inlet and outlet historic plant operation data are collected and stored every minute for most chemical plants, the challenge is to utilize this wealth of data to make more profit. Modelling and optimization of commercial ethylene oxide (EO) reactor and of ethylene glycol production has not yet received sufficient attention in the prior art.

Due to poor understanding and complexity of multiphase catalytic reactions occurring in commercial ethylene oxide reactor, a credible first principle-based model is not available in literature. Availability of large amount of reactor operating parameters data of commercial plants make data driven modelling techniques as a viable alternative approach. The current invention presents an effort to utilize the large amount of process data to find an online EO reactor digital twin framework to convert the information from this data to maximize profit.

EO reactors use silver based catalyst and Ethylene Glycol plant economics greatly depend on catalyst selectivity and activity. Catalyst's selectivity and activity is very sensitive to chloride and other process parameters. It is very crucial to run the EO reactor at optimum operating conditions all the time to maximize the catalyst performance. A minor change in chloride can reduce the catalyst selectivity drastically. Plant engineers could not always detect the optimum chloride in real time basis and thus lose 0.5-1% selectivity on an average throughout the catalyst life. This leads to erosion of potential profits in terms of million USD per year for a moderate size glycol plant. Due to the above reasons, full potential of catalyst performance could not be realized in actual plant. This resulted in huge monetary loss of client plants and catalyst manufacturer.

To tackle the above challenges U.S. Pat. No. 9,892,238 B2 found a technique to detecting abnormal events in an ethylene oxide reaction process using multivariate statistical techniques and artificial neural network. Detecting abnormal events in a running commercial ethylene oxide reactor at incipient stage is very important but optimizing the process parameters to drive the process towards better performance during normal operation is more crucial. In this context, current invention may be considered having been made on the basis of the above US patent as closest prior art. Instead of detecting abnormal event in EO reaction process, present invention focuses on how to optimize the reactor process conditions in real time to achieve higher selectivity of catalyst. One major objective of present invention is to support an engineer in controlling a process of ethylene oxide production. This objective is achieved by finding a real time optimizer of ethylene oxide (EO) reaction system which collects the real time plant operating data from data historian, carry out appropriate analysis of present and future status of the EO reaction system with the help of its in-built model and recommend to the plant engineers appropriate corrective and preventive actions so that performance improvement of reactor is realized in actual plant. The present invention proposes a method of estimating a selectivity and/or activity of a catalyst in an ethylene oxide (EO) reactor. In other words, a calculation based on measurement values is automatically performed in order to determine selectivity and/or activity of the catalyst. The present teaching therefore is embodied by means of a computer implemented invention. The invention is related to a process performed in a chemical reactor in which ethylene oxide is produced. The ethylene oxide reactor can be configured as known in the prior art. Generally, the hardware and the functional parts, the sensors and actuators of the reactor fully correspond to setups of known ethylene oxide reactors. According to the invention, in a first step process data of the process for EO production are acquired. The process data can be measured or calculated data. They can refer to historical states and modes of the reactor and alternatively or additionally to current process data. In other words, the process data can be retrieved from a database or from sensors arranged in a real ethylene oxide reactor, or both. In a second step, based on the process data, a parametric coefficient is predicted. In other words, a computer program, which will be defined further below, is used for automatically determining the parametric coefficient based on the process data. The key performance parameters (examples of process data) for an EO reactor can refer to catalyst selectivity, reactor temperature, ethylene oxide production rate or the like. In a third step, the-parametric coefficient is used for calculating the selectivity and/or activity of the process data. In other words, the selectivity and/or activity of the catalyst is evaluated based on a parametric coefficient, which has been determined from acquired process data or with respect to a certain mathematical function. As a result, measuring selectivity by a distinct sensor or calculating the selectivity from an accumulated amount of ethylene oxide is obsolete.

Based on the result of the calculation a signal for (automatically) adjusting parameters of the process can be generated in an ultimate step. The signal can represent a recommendation or a control signal for adjusting the parameter of the process in order to adapt the process for ethylene oxide production in a real reactor. In other words, the signal optionally is automatically taking effect on the parameter of the control unit of the reactor or at least represents a recommendation to technical staff of a reactor for adjusting the parameter. As a result, the invention can keep the process at best possible conditions for an optimum catalyst selectivity and activity, best possible performance and low costs. All historical information regarding a certain reactor can be regarded in real time when determining the parametric coefficient from the acquired process data and the process can be controlled even by non-experienced engineers, who only have to follow the generated signal for adjusting the parameter or may have to agree/admit application of the automatically adjusted parameter to the process.

The dependent claims relate to advantageous embodiments of the present invention.

The process is characterized by numerous parameters and properties, which can be described by the process data cited above. The process data can comprise current sensor data. These can originate from sensors within the ethylene oxide reactor and characterize the current state of the ethylene oxide production. Additionally or alternatively, a change in selectivity and/or inlet moisture and/or inlet ethylene oxide and/or inlet ethane and/or inlet oxygen concentration and/or carbon dioxide concentration and/or total chloride concentration and/or other parametric coefficients can be determined by sensors of the ethylene oxide reactor. In other words, any of the aforementioned or an arbitrary combination of the aforementioned can be acquired by means of sensors and/or a calculation based on process data. The acquisition of the process data can be performed automatically, in particular periodically and/or on demand. Acute demand can arise from the need for adjusting certain parameters of the process, since best possible information regarding selectivity and/or activity of the catalyst of the ethylene oxide reactor now is required for making best suitable adjustments.

The process data for example can be captured by flow sensors and/or pressure sensors and/or temperature sensors and/or an online analyzer sensor of the ethylene oxide reactor. Other suitable sensors can be employed for deriving the above-mentioned process data.

One preferred way of determining the parametric coefficient from the acquired process data involves calculating the parametric coefficient by means of one artificial intelligence algorithm or two artificial intelligence algorithms, respectively. In other words, the acquired process data can be examined or fed into the artificial intelligence algorithm, which represents a digital clone of the ethylene oxide reactor. The AI algorithm can for example continuously surveil the process data of the ethylene oxide reactor, determine parametric coefficients from the acquired process data and calculate a selectivity and/or activity of the catalyst from the parametric coefficient. The AI algorithm can be understood like a digital clone of the ethylene oxide reactor and can continuously learn the dependencies of the selectivity and/or activity of the catalyst from the parametric coefficient and the acquired process data. In this way, the artificial intelligence algorithm can predict the selectivity and/or activity of the catalyst from current parametric coefficient, which is determined based on the acquired process data. In this way, a future value of the selectivity and/or activity of a catalyst, which e.g. represents the need for replacing the catalyst, can be communicated to an operator of the reactor via a signal or a message. For example, the message can be sent to a (wireless) communication device of the operator in order to notify him of the need for replacing the catalyst within a predefined period. The inventors have surprisingly found out that a combination of an first principle based and a genetic programming provides best possible accuracy of prediction of selectivity and/or activity of the catalyst from the parametric coefficient.

In particular, the determination of the parametric coefficient from the acquired process data can be formed by applying a genetic programming model and/or a kinetic based detail phenomenological model to the acquired process data. In particular, a combination of the genetic programming model and the kinetic based detail phenomenological model have proven to provide optimal prediction results.

Further preferably, the determination of the parametric coefficient from the acquired process data can be performed by applying a combination of a genetic programming model and a second model of the process to the acquired process data. The second model of the process can for example be a first principle-based kinetic model. This first principle-based kinetic model in particular interacts with the genetic programming model when deriving the parametric coefficient from the acquired process data.

If the model of the process is a first principle-based kinetic model, a prediction error of the model can be minimized by an artificial intelligence-based data driven model

Preferably, the acquired process data can comprise internal reactor data, that is data of the reactor which are acquired by sensors within the reactor. The internal reactor data can in particular comprise an age of a catalyst and/or a temperature and/or a pressure within the ethylene oxide reactor. The age of the catalyst can for example be determined by a timer, which has been started when replacing the catalyst most recently. Alternatively or in addition to the internal reactor data, external reactor data can be acquired as process data. The external reactor data can in particular comprise an ethylene oxide stripper bottom temperature and a pressure and/or cycle water system data, in particular cycle water flow and temperature and/or carbon dioxide regenerator bottom temperature and/or carbon dioxide removal system data, in particular carbonate flow and/or density and/or temperature. These internal and external reactor data provide a best possible basis for automatically determining a parametric coefficient for calculating the selectivity and/or activity of the catalyst.

For even improving the present invention the method can involve a step of training a genetic programming by using sensor data of the process for ethylene production. In other words, the sensor data of the ethylene oxide reactor are involved when improving the genetic programming algorithm used for determining the parametric coefficient from the process data. Applying this new and improved genetic programming model even provides better results when calculating the selectivity and/or activity of the catalyst from the parametric coefficient.

Optionally, the prior model and the recently found model can be compared to each other in order to refine the modelling technique and improve the model building process. Thus, evolution of the digital twin EO reactor model is positively affected.

Further details, advantages and features of the present invention are disclosed in the following description of embodiments according to the drawing. Therein,

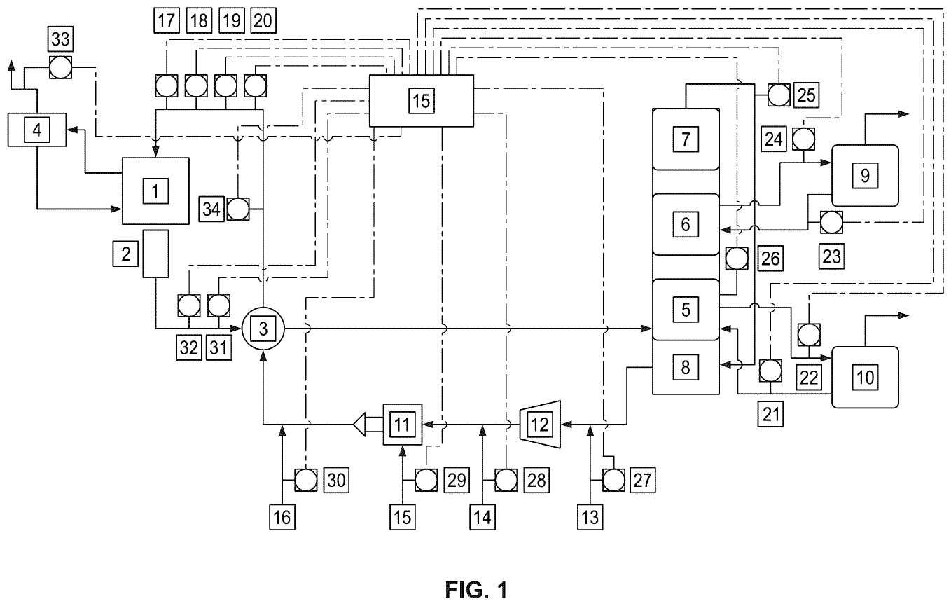

FIG. 1 shows a schematic of an ethylene oxide reactor and associated unit;

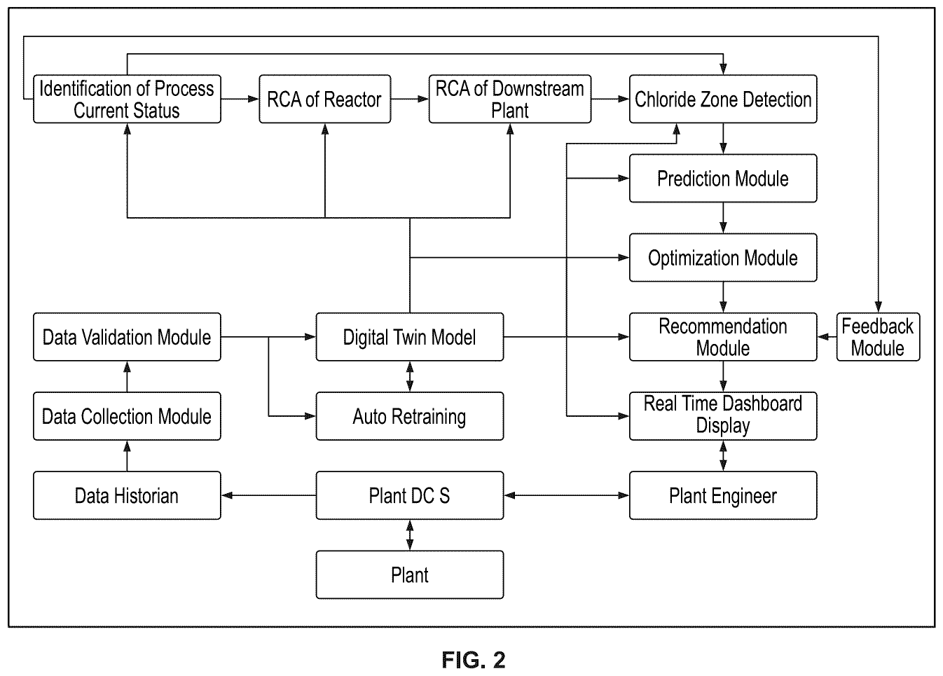

FIG. 2 shows the basic structure of the ethylene oxide reactor digital twin application software according to an embodiment of the present invention;

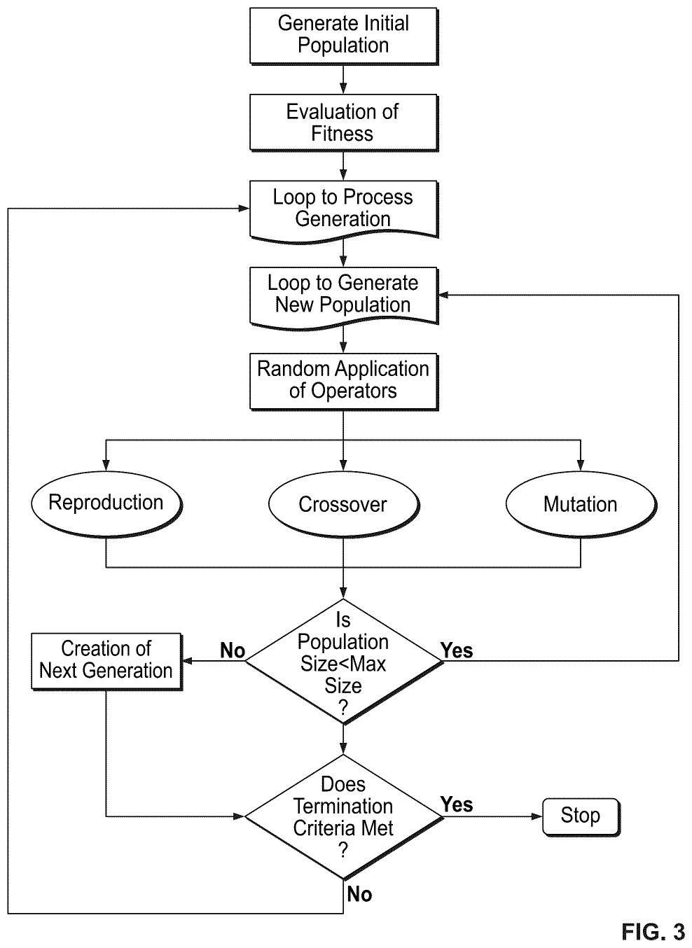

FIG. 3 shows an exemplary algorithm of genetic programing (GP) as used in an embodiment of the present invention;

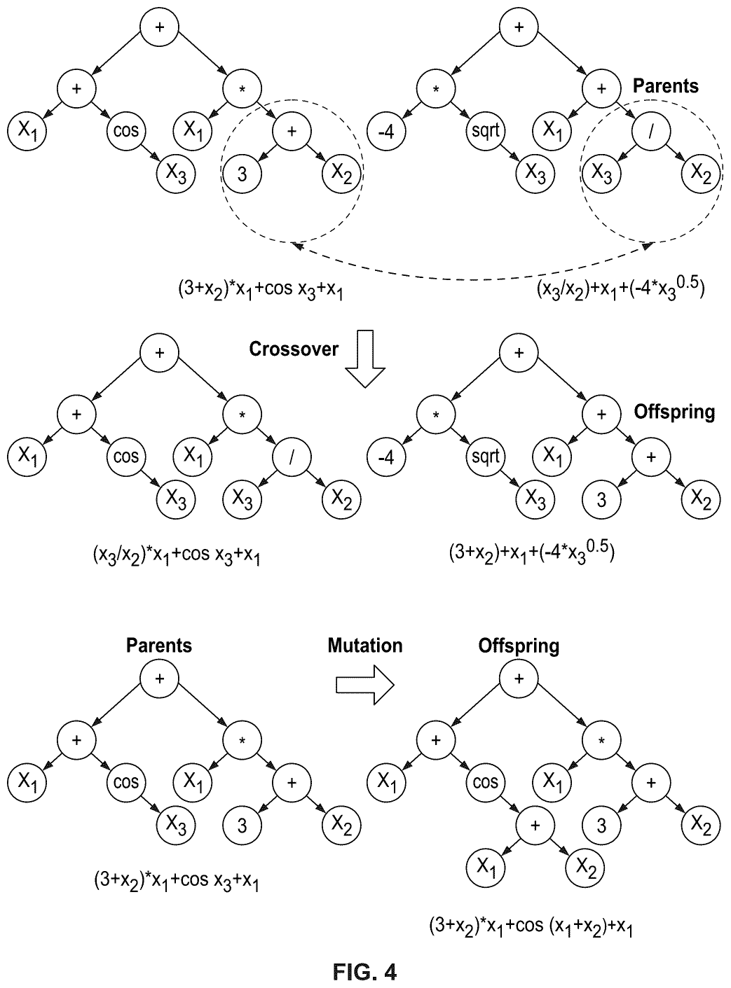

FIG. 4 shows the process of reproduction, cross over and mutation steps within genetic programing;

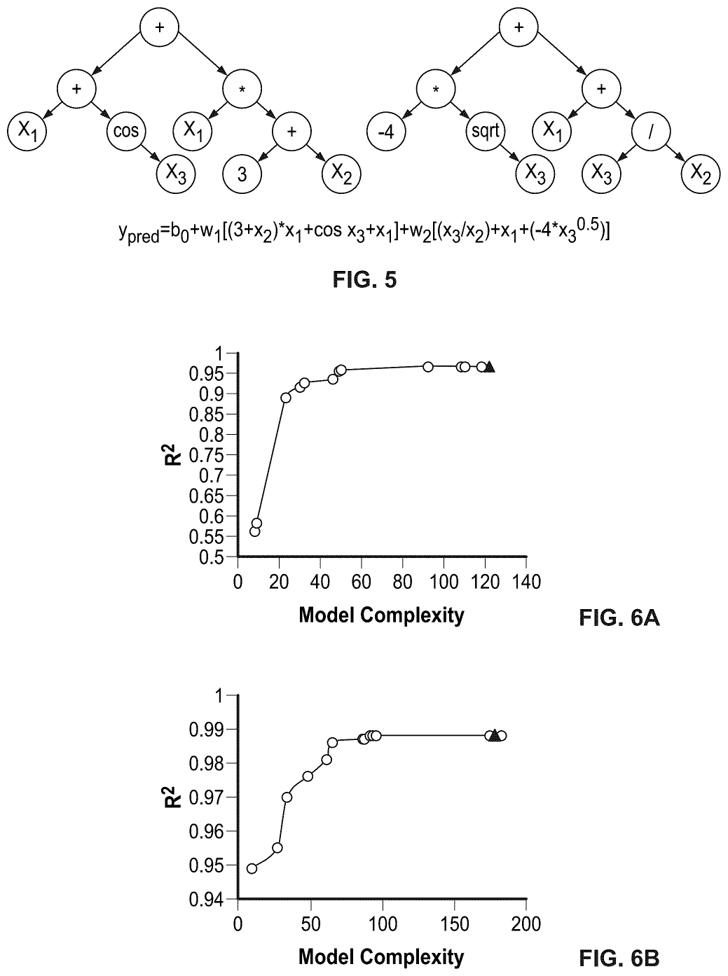

FIG. 5 shows a typical multi-gene genetic programing (MGP) model;

FIG. 6A show the influence of model complexity versus the prediction accuracy;

FIG. 6B show the influence of model complexity versus the prediction accuracy;

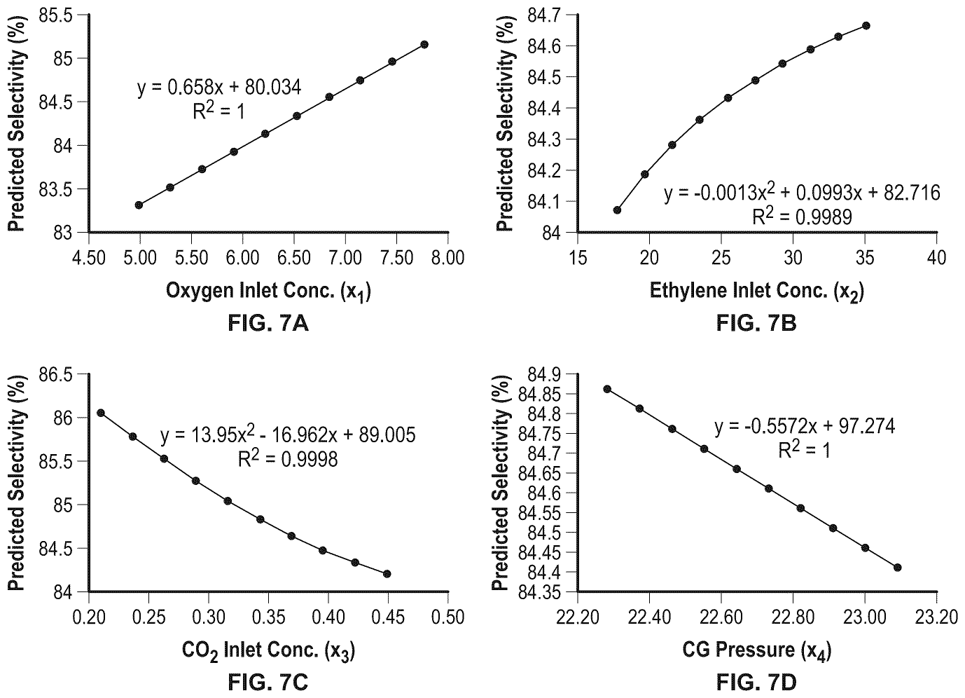

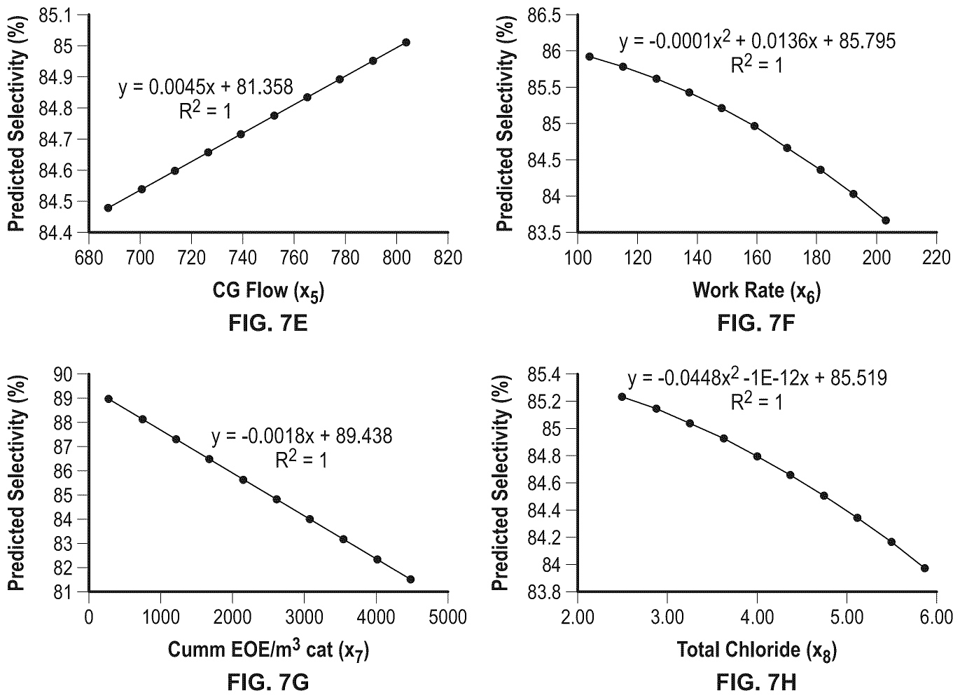

FIG. 7 show selected relationships between parameters of the inventive process;

Specifically, against the predicted selectivity %:

FIG. 7A shows the O2 inlet concentration;

FIG. 7B shows the ethylene inlet concentration;

FIG. 7C shows the CO2 inlet concentration:

FIG. 7D shows the CG pressure;

FIG. 7E shows the CG flow;

FIG. 7F shows the work rate;

FIG. 7G shows the cumulative EOE/m3 cat

FIG. 7H shows the total chloride.

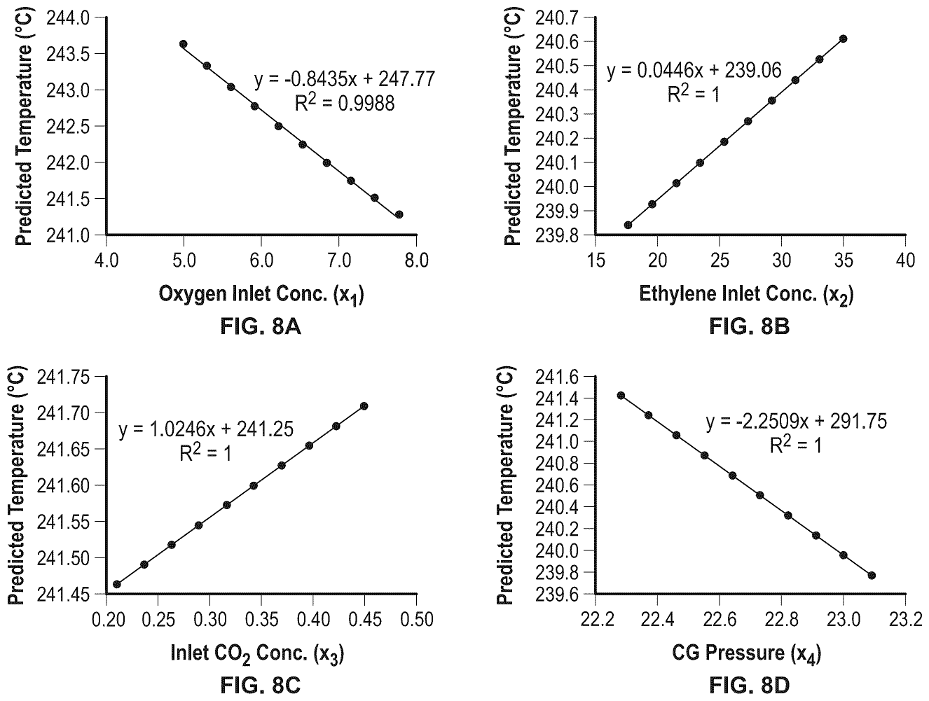

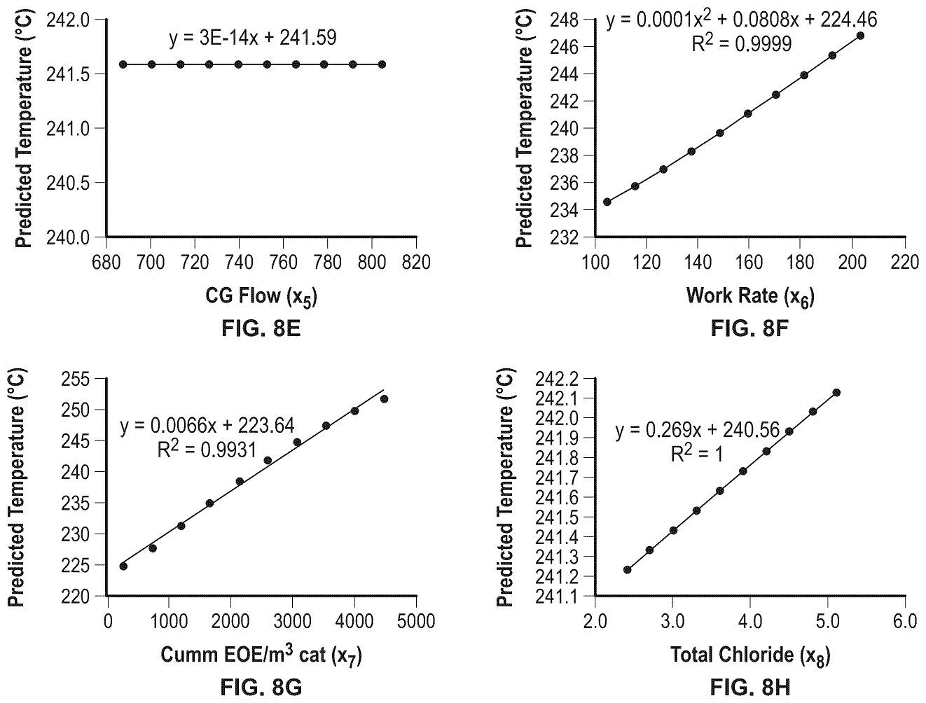

FIG. 8 show selected relationships between parameters of the inventive process;

Specifically, against the predicted temperature:

FIG. 8A shows the O2 inlet concentration;

FIG. 8B shows the ethylene inlet concentration;

FIG. 8C shows the CO2 inlet concentration;

FIG. 8D shows the CG pressure;

FIG. 8E shows the CG flow;

FIG. 8F shows the work rate;

FIG. 8G shows the EOE/m3 cat;

FIG. 8H shows total chloride

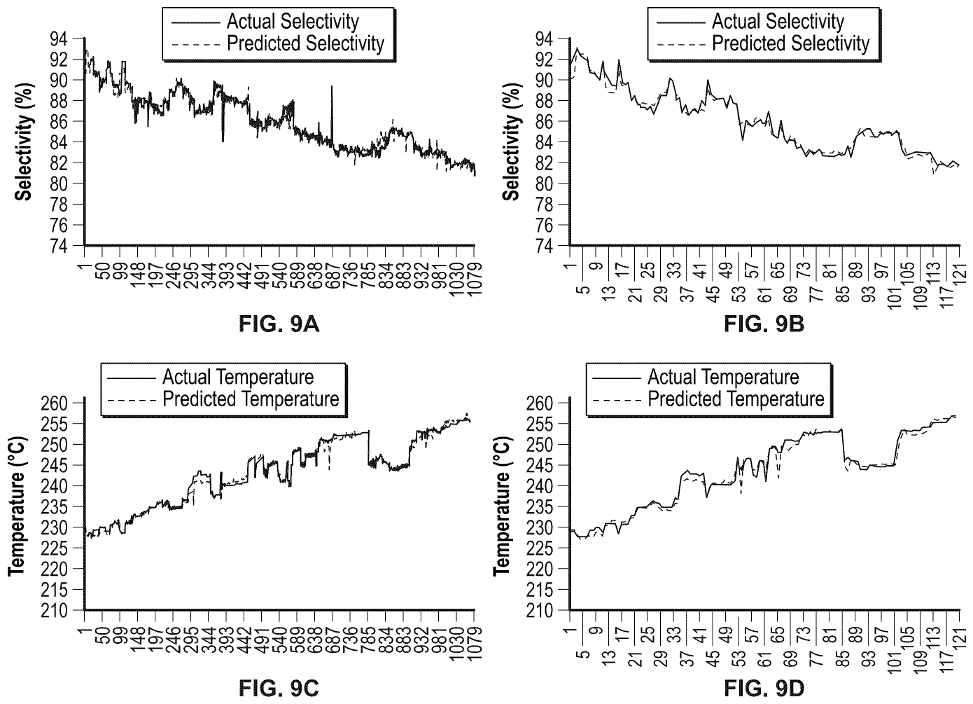

FIG. 9 shows the inventive model's prediction performance on training and test data for temperature and selectivity;

### Specifically,

FIG. 9A and FIG. 9B show the actual selectivity against the predicted selectivity;

FIG. 9C and FIG. 9D show the actual temperature against the predicted temperature.

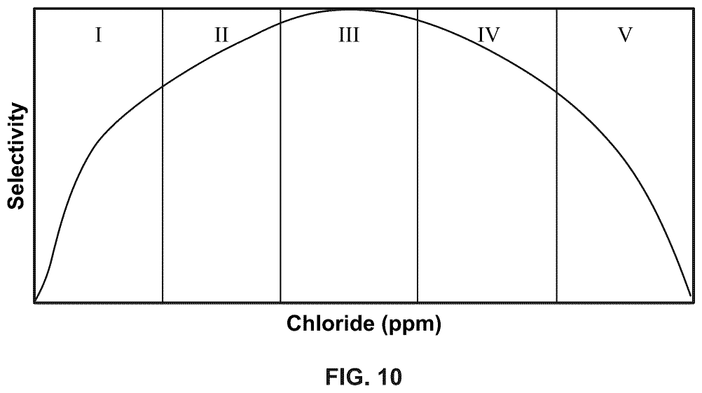

FIG. 10 shows an exemplary division of the selectivity and chloride curve;

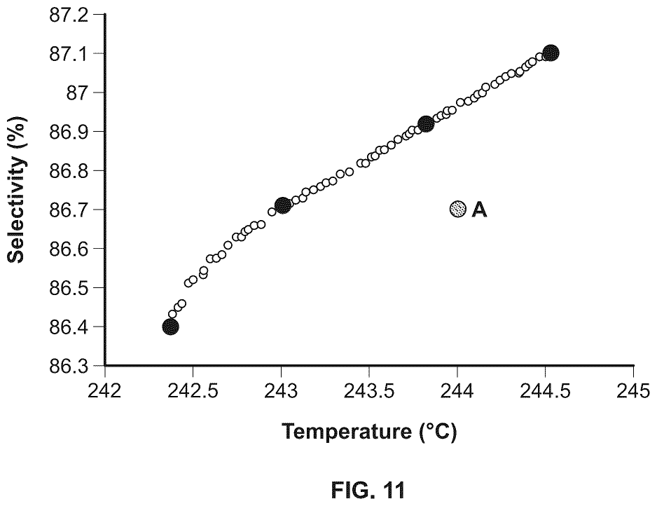

FIG. 11 shows an exemplary pareto diagram for an exemplary non-dominated optimal solution; and

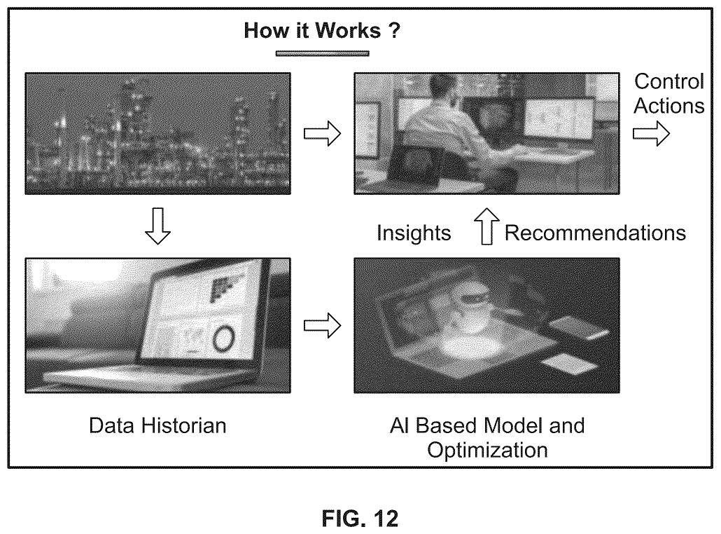

FIG. 12 shows a principle flow chart depicting an embodiment of the present invention.

Advantageously, in the present invention a digital twin application of the commercial EO reactor is built to mimic the real plant. This application can run in real time on a digital processing unit (e.g. a desktop computer) and collect plant data historic data (also named “historian” in this disclosure) e.g. at a regular interval of e.g. one hour. The software will collect the data in real time and carry out all the analysis in order to collect information for efficiently controlling the plant. With the help of its in-built AI based model of any other suitable algorithm, it will assess the current status of the plant, carry out root cause analysis for performance deterioration of catalyst (if any), predict the future performance and optimize the reactor operating parameters to increase catalyst performance. Moreover, this application will generate and communicate the decision to change chloride and other process parameters to respective control devices (valves, dosage systems, heating or pumping devices etc.) in order to establish a best performance for the process immediately or in order to communicate suggestions and/or target values for the parameters (e.g. on a dashboard and/or infographics) to an engineer.

### Example of a Process to be Monitored and Optimized

The EO reaction process of commercial glycol plant in this disclosure can be applied to the hardware and implemented as software in a plant or process exemplary described in US patent 2018/9,892,238 B2. Oxidation of ethylene to produce ethylene oxide (also named “EO” in the following) is an important reaction in the petrochemical industry for synthesis of glycol, for instance—Commercially EO is produced in shell and tube type EO reactors by reacting oxygen and ethylene at high temperature and pressure in presence of silver-based catalyst. The oxidation of ethylene involves a main reaction producing EO (epoxidation reaction) and an undesirable side reaction producing carbon dioxide or C02 (combustion reaction).

Desired Main Reaction (epoxidation reaction)

Ethylene+oxygen→Ethylene oxide

Undesired Side Reaction (combustion reaction)

Ethylene+oxygen→carbon dioxide+water

The performance of the reaction is measured by selectivity, which is calculated by the percentage of ethylene used to produce EO as compared to total ethylene used to produce EO and C02. Indirectly, selectivity measures the extent of the first reaction as compared to the second reaction. Selectivity has a profound effect on the efficiency and hence the overall economics of the glycol plant.

$$\\ Catalyst selectivity = \frac{Moles of EO produced}{Moles of ethylene consumed}$$

An EO reactor may be built like a shell and tube heat exchanger. Silver catalyst may be put as a fixed bed in a tube side. Water is circulated through the shell side to remove the heat of reaction as both the reactions are exothermic. The conversion of ethylene to EO is very low, therefore ethylene and oxygen are recycled back as illustrated in FIG. 1. FIG. 1: Schematic of EO reactor and associated unit shows a typical ethylene oxide reactor along with downstream ethylene oxide scrubber, carbon dioxide contactor and wash section. A mixture of gas, namely cycle gas is fed to the ethylene oxide reactor 1 from top and continuously pure oxygen 15, ethylene 16 and methane 13 is fed to the cycle gas system as shown in FIG. 1. The reactor may be built like a shell and tube heat exchanger wherein high selectivity catalyst pellets may be loaded as packed bed at the tube sides. Coolant is circulated through the shell side to remove heat of reaction and thus produce steam in steam drum 4. Ethylene and oxygen are partially reacted in the catalyst bed producing ethylene oxide (EO), carbon dioxide and water. Reactor outlet gas is further cooled in gas cooler 2 and gas-gas exchanger 3 and fed to the ethylene oxide scrubber 5 to absorb EO by water. The absorbed EO is stripped from the cycle water in EO stripper 10 and lean cycle water recycled. Cycle gas from the EO scrubber top is fed to the CO2 contactor 6 to absorb carbon dioxide by carbonate solution and finally to wash section 7 to wash any residual carbonate particles. Cycle gas from the top of the wash section is fed to knock out drum 8 to remove any liquid and finally recycled back to the EO reactor via cycle gas compressor 12. The CO2 rich carbonate is regenerated in Regenerator 9 and lean carbonate solution is recycled. A chloride activator 16, preferably ethylene dichloride EDC or ethyl chloride EC is continuously fed to the cycle gas in a small quantity which acts as an activator and selectivity promoter in EO reaction system—A small amount of chloride (in ppm level) coming from activator is sufficient to increase the selectivity and activity of catalyst-EDC or EC inhibits the combustion reaction, i.e., the second reaction to a greater extent than the epoxidation reaction, i.e., the first reaction. In this way, EDC or EC promotes the selectivity for EO. Less than optimum quantity of inhibitor reduces selectivity and produces more carbon dioxide. Thus, the optimum value of inhibitor concentration at reactor inlet is crucial for maximizing EO production. Over-dosing and underdosing of activator can reduce the catalyst selectivity drastically and lead to an abnormal situation. Optimum dosing of activator is thus necessary for maintaining highest selectivity all the time. However, optimum dosing rate is not constant and can vary with catalyst age, chloride losses from the system and reactor temperatures, for instance. Because of the complex dynamics of the process, it is very difficult to calculate the optimum chloride dosing rate theoretically. High selectivity catalyst is very sensitive to the chloride-dosing rate and any deviation from the optimum dosing rate has an adverse effect on selectivity and overall economics of the process.

There are two associated processes, namely, cycle water process and CO2 removal process which are interconnected with EO reaction process. The EO rich cycle water after absorbing EO from cycle gas in EO scrubber fed to EO stripper. EO is stripped off from cycle water in EO stripper by applying heat and then EO free cycle water then again recycled back to scrubber to absorb EO again. This complete the cycle water closed loop. Similarly, CO2 rich carbonate solution from CO2 absorber fed to regenerator where CO2 is removed from carbonate solution by application of heat. CO2 free carbonate solution then recycled back to CO2 scrubber again to absorb CO2 from cycle gas and thus completes the closed carbonate flow loop.

Typically, there may be in the region of 20 independent and 35 dependent variables associated with such a process, not all of which are shown in this example. Independent variables measured include the cycle gas inlet composition (nine components, such as oxygen, ethylene, methane, ethane, carbon-dioxide, water, ethylene oxide, nitrogen and argon measured by online analyser 18), cycle gas flow 17, pressure 20, coolant temperature 19, chloride concentration at reactor inlet gas i.e., different chloride species such as ethylene di-chloride, ethyl chloride, vinyl chloride, methyl chloride, allyl chloride measured by online chloride analyser 34, methane flow 27, ethylene flow 28, oxygen flow 29, EDC flow 30, EO scrubber top temperature 26, wash tower top temperature 25 etc. Examples of dependent variables include cycle gas outlet composition 9 components as specified above measured by analyser 32, selectivity of catalyst 31, EOE production (calculated), steam generation in steam drum 33 etc. All the sensors and meters are interfaced to an online real time data historian 15 as shown in FIG. 1.

FIG. 2 shows an exemplary depiction of the basic structure of the EO reactor digital twin (DT) application software. At the heart of Digital twin application is the process plants with all of its Proportional integral and derivative (PID) controllers. Plant is connected with DCS/PLC systems, which scan the plant at a rate of e.g. several times a minute or second, preferably about every second. Digital twin module starts at the supervisory level above DCS. DT online software package normally interacts with DCS via data historian and human interface. Following sections gives an overview of various modules of digital twin application and their common features.

The basic structure of the EO reactor digital twin (DT) application software is as follows:
1. Data Collection Module: This module collects real time data from plant data historian at an interval of e.g. 1 hr.
2. Online Data cleaning and data validation module: This module cleans any faulty data or outliers. It also validates the accuracy of measure data with a detailed validation calculations based on mass, energy and component balance.
3. Digital twin model: This is the heart of the whole application. An artificial intelligence based mathematical model of EO reaction process is at the core of digital twin application.
4. Identification of current status of EO reaction process: Based on current value of process parameters and with the help of digital twin predictive model, this module identifies where the process stands currently and how is the current performance of EO catalyst.
5. Root cause analysis in EO reactor: This module determines the root cause analysis of catalyst performance deterioration in the past, e.g. last 24 hrs, if any.
6. Root cause analysis of downstream plant: This module identifies the root causes of performance degradation, if any of cycle water system and CO2 removal system
7. Current Chloride zone detection: Based on logic of catalyst characteristics this module determines the current chloride zone.
8. Prediction module: Based on current status of the plant and with the digital twin predictive modules, this module predicts the catalyst performance and production performance in the future, e.g. for next 24 hrs.
9. Optimization module: This module optimizes the process parameters of EO reactors on real time basis to improve the catalyst performance.
10. Recommendation module: This module summarizes what action plant engineers need to take to improve catalyst performance. It takes helps of prediction and optimization modules to give real time recommendations e.g. in fixed time intervals, such as every hour.
11. Feedback module: This module tracks the real time feedback of plant for past recommendations and suggests any corrective actions to recommendation modules.
12. Auto retraining module: this module tracks the digital twin models predictive accuracy and execute an online auto retraining with most recent data to update the models. This ensures digital twin model remains up-to-date and with high prediction accuracy all the time of catalyst life.
13. Real time dashboard displays: This is an interface between digital twin algorithms and plant engineers. All the past and future trends of process parameters, results of root cause analysis, and real time recommendations are displayed in this dashboard with various infographics.

In the following, the detail function of each module and related calculations are discussed.

### 1. Data Collection Module

It starts with data collection module. Purpose of this module to collect real-time data from plant data historian. Usually data collector module directly collect data at an interval of one hour from plant data acquisition systems usually installed in glycol plants (Like IP21, PI systems etc.). Data acquisition system usually connected with plant DCS, which in turn connected with different flow, temperature, level, pressure transmitters and also online analyzers in actual plant. Key process parameters in and around EO reactor, wash tower, cycle water system and carbon-dioxide removal system are identified in-priori and data collection system is continuously collecting real time data of these process parameters in fixed time intervals, e.g. every hour.

### 2. Online Data Cleaning and Data Validation Module

As this digital twin application module is collecting real time data from data historian and do a background model-based calculations; it is very critical that it collects and interprets the accurate data. Data accuracy is very crucial in such type of online optimization module.

However, industrial EO plant data usually can have following concerns:
Data may go as outlier due to malfunction of measuring transmitters, data transmission or data collection error, noise in the data etc.
Some transmitters show shifting of zero and span. This will shift the collected data and this introduce measuring error.
Due to its inherent complexity, the malfunction of online analyzer is very common in EO plant due to sample line problems, inaccuracies in online analyzer (gas chromatograph and/or mass spectrometry), interference effect in analyzer etc.

Hence, for a real world running glycol plant, inaccuracies, outliers, spike and drifting of measured data is very common and expected. This inaccurate data collection is dealt with following ways in present invention:

### 3. Data Cleaning Module

This module statistically detects and cleans the outliers in data as soon as collected.
Any data going beyond its average value by a certain value, e.g. ±3 standard deviations value is considered as outlier and assumed to be result of abnormality in either transmitter, analyzers or data collection systems etc. Standard deviations of each process parameters are calculated from its historical data collected over a predefined period (e.g. last 2 years).
These outlier data are identified every hour as soon as it gets collected, immediately generate warning alarm in the online application and not taken inside the calculation module. These outlier data are replaced with last good value and a warning log records all such incidents for future verifications.

### Pseudo Code of Data Cleaning

1. Calculate average value and standard deviations of each parameter from its past predefined time interval, e.g. 2-year historical data
2. Designate the current measured data as good value and accept the current value as it is if the current data fall between average value by a certain value, e.g. above mentioned ±3 rd deviations range
3. Designate the current measured data as bad value and replace the current value with last good value if the current data fall beyond average value by a certain value, e.g. ±3 standard deviations range

### 4. Data Validation Module

If data are drifted and not beyond their average by a certain value, e.g. ±3 standard deviations level, then data are put in a detailed validation calculation module to check its accuracy.

In glycol plants, most of the process parameters are interrelated and they are moved together in certain fashion. Also, EO reactor system has many redundant measurements, i.e. there may be two or more transmitters measuring the same flow. These redundant transmitters are kept strategically during design phase so that redundant transmitters readings can be used in case original transmitters malfunctions or stop working.

This interdependence of various process parameters and redundant measurements forms the basis of data validation calculations.

Mainly 3 performance parameters namely selectivity, temperature and equivalent ethylene oxide (EOE) production from reactor need to be verified and validated. All other reactor parameters can be calculated from them.

### Validation Calculations of Selectivity

1. Validity based on component balance:
   Component balance equations are derived from reaction stoichiometry and component mass balance across reactor and can be used to validate the selectivity indications.
   Following equations are used to calculate selectivity based on component balance:

S1=100*Delta EO/Delta C2H4

S2=200*Delta EO/(2*Delta EO+Delta CO2)

S3=600*Delta EO/(5*Delta EO+2*Delta O2)

S4=(600*Delta C2H4−200*Delta O2)/(5*Delta C2H4)

S5=(400*Delta O2−600*Delta CO2)/(4*Delta O2−5*Delta CO2)

S6=(200*Delta C2H4−100*Delta CO2)/(2*Delta C2H4)

Where Delta represents difference between reactor outlet and reactor inlet concentrations for the particular component. Equations S1-S6 represent same catalyst selectivity calculated in different ways. All the above six equations are derived from reaction stoichiometry and based on fundamental chemistry.
2. Validity based on mass balance across reactor:

$${S}_{11} = \frac{2800 EOE}{44 Net Ethylene flow} \\ {S}_{33} = \frac{600 ( \frac{EOE}{44} )}{5 ( \frac{EOE}{44} ) + 2 ( \frac{Net Oxygen flow}{32} )} \\ {S}_{44} = 120 - 35 ( \frac{Net Oxygen flow}{Net Ethylene flow} )$$

S11, S33 and S44 are catalyst selectivity calculated by different flows.

Where Net Ethylene flow and Net Oxygen flow are the net oxygen and ethylene consumption in reaction and calculated as follows:

$$Net Ethylene flow = Ethylene flowmeter measurement ( \frac{MT}{hr} ) - compensation for [ ethylene concentration increase in cycle gas + cycle gas pressure increase + ethylene purity ]$$

$$Net Oxygen flow = Oxygen flowmeter measurement ( \frac{MT}{hr} ) - compensation for [ o xygen concentration increase in cycle gas + cycle gas pressure increase + oxygen purity ]$$

### 3. Validity Based on Heat Balance Across Reactor

Both the epoxidation and combustion reaction occurred in EO reactor are exothermic and generate heat. This heat is taken away by water circulation and steam generation in shell side of reactor and sensible heat by cycle gas. Since the heat of reaction of Co2 reaction is much more than EO reaction, heat generation varies as per selectivity. This fundamental heat balance concept is utilized to calculate the selectivity from steam generation data and cycle gas temperature rise across reactors.

Following guiding equations are used to calculate steam selectivity.

$$Heat taken away by water = Steam generations from reactor steam drum ( \frac{MT}{Hr} ) * Latent heat of water$$

$$Steam selectivity = \frac{Heat of reaction of first reaction * EOE ( \frac{MT}{hr} )}{Heat generated in reactor}$$

### Basis of Validation Calculations

All the 10 selectivity mentioned above namely [S1-S6, S11, S33, S44 and steam selectivity] ideally should have same value if all the measurements of mass spectrometer [or gas chromatograph], oxygen, ethylene and steam flow meters are correct. In real plant, always there are some measurement errors and all ten selectivity are slightly different and their accuracy can be measured by a term called selectivity spread and defined as follows:

Selectivity spread=[max selectivity-min selectivity] of 10 selectivity calculations

In online digital twin application, a solver was run to minimize the selectivity spread by varying all independent parameters namely delta concentrations of individual components and various flows. Oxygen flow and delta oxygen are taken at their measured value to reduce the multiplicity of solutions.

In this way the true value of concentrations and flows are calculated irrespective of slight error in their measurements.

These calculations involved reading from flow, temperature, pressure transmitters, analyzer readings etc. So even one or two transmitter showing wrong results or drifted then that could be identified by validation calculations involving all other transmitters.

Based on above validation calculations all the EO reactor process parameters are cross checked, validated and recalculated. These re-calculated values are then used for model building and subsequent calculations. Application of detailed validation calculation for EO reactor inside an online optimization model is new.

### Digital Twin Model

To optimize the commercial EO reactor, it is required to model complex systems of industrial chemical reactions as a first step.

Following sections describe the details of model building.

### Background of Model Building and Prior Art

Industrial chemical reactions involved complicated reaction kinetics and thermodynamics. Building a credible phenomenological model for commercial reactors is considered as a very time-consuming difficult task as it demands a deep knowledge of industrial heterogeneous catalytic behavior characterized by mass diffusion, catalyst deactivation, etc. Lack of sufficient knowledge in chemical reaction kinetics actually hinders the optimization of the reactor. In most chemical plants, the reactors remain as black-box, and operation engineers do not try experimentation in running plants due to safety and reliability reasons. This drives non-optimum operation of industrial reactors, which greatly affects the profitability of the plant. So, reactors are considered as unexplored territory of chemical industries. However, a small increment of catalyst selectivity and reaction yield significantly impact the raw material consumption and plant profitability in large-scale operation. [1]

Hence, an easy alternate approach is applying data-driven effective computational technique to build approximate reactor models for these complex reaction systems, which can be subsequently used to optimize the reactor and increase profit.

As huge reactor inlet and outlet historic plant operating data are collected and stored every minute for most chemical plants, the real challenge is to utilize this wealth of data to make more profit. Data is new oil, and certainly, data-driven modelling techniques like artificial neural network (ANN) and support vector machine (SVM) are considered new IC engine of current age. However, as to the chemical industry, ANN and SVM models are not favored by the plant engineers due to lack of their expandability and black-box nature. ANN modelling provides an equation consisting of complex sigmoidal function with several tuning constants, known as weights and biases, and that is why the model suffers from the limitation of explain ability though the prediction capability of ANN model is excellent. Usually, process engineers want manageable equations in differential/algebraic form relating output variables with input features so that they can get more insight to derive benefit. Hence, it is needed to explore alternate computational methods.

Genetic programming (GP), which is a branch of evolutionary modelling techniques, has been recognized by the inventors to have the capability to remove the above drawbacks of ANN and SVM models among chemical problems. Genetic programming (GP), automatically generates nonlinear structured models as closed-form equations relating input and output of the system from available data. Not only GP identify the structure of the equation, but also it estimates the different parameters of the equations so that it accurately predicts the output. At the time of building up MISO (multiple input, single output) models by using GP, the probability of survival of a particular model to its next generations depends on its prediction accuracy and followed ‘survival of fittest’ principles. Recombination of components of survived models continuously takes place to form new model aiming at increasing predictability in each generation.

In literature, the breakthrough in GP can be seen in the late 1980s with the experiments of Koza [2] on symbolic regression. The versatility of GP algorithm is proved by Koza and Rice [3] by applying in diverse fields of robotics, games, control, etc. Both dynamic and steady-state modelling of binary distillation column and twin-screw cooking extruder respectively were performed by Willis et al. [4] with the help of GP. Gray et al. [5] implemented GP-assisted dynamic modelling to generate continuous discrete models using MATLAB Simulink combination with sets of equations. Mckay et al. [6] demonstrated the application of GP in solving nonlinear test problems of vacuum distillation column, reaction system. Lakshminarayanan et al. [7] combined principal component analysis with GP to build up nonlinear models and applied the same in product design. Grossman [8] applied GP for the generation of empirical models in process system. Grossman and Lewin [9] presented an approach to automate the design of nonlinear MPC (NMPC) using a GP-driven, model-based control approach.

Multi-gene genetic programming (MGP) [10, 11] is one of the robust variants of GP and claims to be more effective than GP in nonlinear modelling. MGP is designed to generate mathematical models of predictor response data that are “multi-gene” in nature, i.e., linear combinations of low-order nonlinear transformations of the input variables. The conventional GP is mainly on the basis of evaluation of a single tree (model) expression.

In multi-gene approach, several individual genes are combined to construct a single one. [11] It has been proved that MGP regression is capable of providing more accurate and computationally efficient model in comparison to conventional GP. [10, 11] It can be seen in many cases that MGP performed better than other machine learning methods, viz. ANN, SVM in terms of predictability and model simplicity. [12-15]

Despite the remarkable prediction capabilities of the MGP approach, applications of this method to chemical reaction engineering tasks in commercial reactors are conspicuous by their near absence. The reason-as discussed above-is to be seen in the clear preference of the engineers and stakeholders for deterministic approaches and control algorithms. Moreover, complexity of reactors make modelling a challenging task.

Additionally, impurities in the starting material could have had disastrous consequences, if not adequately reflected in and considered by the model. Finally, the question of responsibility and liability for economical damage and personal injury may have prevented GP from being considered in chemical reaction engineering tasks. In the present invention MGP modelling approach is applied to build model commercial chemical ethylene oxide (EO) reactor.

One major objective of present invention was to find an accurate commercial EO reactor model which is in closed form equation, portable, explainable, deployable in real time application and can be used by the plant engineers to gain insight of the process and valuable support for adjusting parameters and concentrations.

### 5. Model Building of Ethylene Oxide Reactor Background

Due to poor understanding and complexity of multiphase catalytic reactions occurring in commercial ethylene oxide reactor, a credible first principle-based model is not available in common chemical handbook and similar literature. Availability of large amount of reactor operating parameters data of commercial plants make data driven modelling as a viable alternative approach. The current work presents an effort to utilize the large amount of process data to find a framework to convert the information from this data to maximize profit.

### 6. Production Objectives

Because of catalyst poisoning and sintering effect, the catalyst selectivity decreases gradually with the age of the catalyst, and the catalyst selectivity remains around 90% at the starting of operation (SOR) and becomes 80% at the end of the operation (EOR) (usually after 2-4 years). The average catalyst life of present generation catalyst is around 24-48 months. To maintain the plant production at target while selectivity is decreasing, the temperature of the reactor is gradually increased over the age of the catalyst life (e.g., from 225° C. in SOR to 275° C. in EOR) to increase the reaction rate. Catalyst selectivity determines the profitability of plant as higher selectivity means less consumption of expensive ethylene and oxygen to produce same amount of EO i.e., less production cost. On the other hand, reactor temperature determines catalyst age. Faster rise of reactor temperature means loss of catalyst age and incurring fixed cost of catalyst sooner by replacing the catalyst and taking plant shutdown for that. In commercial plant, two prime production objectives are optimizing the reactor operating parameters in such a way so that selectivity maximization and minimization of reactor temperature (i.e., maximization of catalyst longevity) are achieved simultaneously while maintaining plant production target.

### 7. Genetic Programming: At A Glance

GP is a branch of metaheuristic symbolic optimization technique which generates equations for solving a problem using the ‘survival of the fittest principle’ of Darwinian natural selection.

The general form of the reactor model to be obtained is given as below (Equation 4)

y=f(X, β) (4)

where y indicates the process output variable (Selectivity or catalyst temperature); X is the N-dimensional vector of input variables like flow, pressure, inlet concentration of various raw materials etc. (X=[x1, x2, . . . , xn, . . . , xN]T), and f denotes a nonlinear function whose parameters are defined in terms of a P-dimensional vector, β[β1, β2, . . . , βK]P. If industrial data of input and output variables are given, GP algorithm tries to best fit the data by changing its functional form and parameter vector β.

### 8. Executional Steps of Genetic Programming

The algorithm of GP has been illustrated in FIG. 3. The executional steps of GP are mentioned as below:

Step 1 (Initialization): In the first step, GP algorithm creates random equations to fit the data defined in Equation 4. These equations commonly called population of strings (chromosomes) representing candidate solutions. Basically, a population member consists of functions and terminals combined in hierarchical manner, which is termed as tree. The function set may contain algebraic operators, Boolean logical operators. The terminal set may consist of variables, numerical and logical constants. A typical tree can be seen in FIG. 4.

Step 2 (Generation): This step is iterative procedure to generate the population with high fitness value and consists of the following sub-steps:
a) The fitness of each population is evaluated using a pre-specified fitness function. Coefficient of determination (R2) dependent or error dependent fitness function can be used for this purpose. The higher the value of R2 or the lower the value of error, the better the fitness of a particular population. In this invention root mean square error (RMSE) was considered as error parameter.
b) Select individual equations from the population with the help of probabilistic determination of fitness
c) Create new individual equations with the help of genetic operators such as:
   (i) Reproduction: During reproduction, the algorithm copies the existing population into new generation without any change
   (ii) Crossover: In crossover, off-spring is produced by the interchanging of chromosomes of parent generation.
   (iii) Mutation: In mutation, replacement of existing elements in offspring with other elements takes place.

The reproduction, crossover, and mutation steps are illustrated in FIG. 4.

Step 3 (termination): When the termination criteria are met, the best program will be the approximate solution of the problem.

### 9. Multi-Gene Genetic Programming (MGP)

The conventional GP is generally used for symbolic regression of a given input output datasets but often, conventional GP suffers from limitation of lesser accuracy. On the contrary, multigene approach enhances the accuracy of the model in higher extent. Multigene Genetic Programming (MGP) makes weighted linear combination of smaller GP trees or genes in order to maximize fitness. In MGP, predicted output variable is the summation of bias term and weighted output of individual trees (genes). The predicted variables in MGP can be expressed as follows (Equation 5):

$${y}_{pred} = {b}_{0} + {\sum }_{i = 1}^{N} {w}_{i} {g}_{i} \qquad ( 5 )$$

where y_{pred} is the predicted output, bₒ is the bias term, gᵢ is the genes or trees and wᵢ is the corresponding weightages. Similar to linear regression, the weightages and bias are determined by least squares method. Therefore, MGP captures the nonlinear behavior through the small trees and also applies classical linear regression methodology.

A typical MGP model has been pictorially represented in FIG. 5. Here predicted output variable is expressed in terms of three input variables (x1, x2 and x3). In FIG. 5, the two genes have been shown which are linearly combined with each other to form an MGP model. The b0, w1 and w2 are the bias and weightages which are calculated at the time of training by using least square method. User in practice specify the maximum number of genes that will be used and the maximum depth of a particular tree. It is obvious that more the depth and number of genes, more will be accuracy but the complexity will also be more.

### 10. Selection of Input and Output Variables for Modelling

Since selectivity and reactor temperature has a large impact on plant profitability, these two parameters are kept as output variables. All the reactor operating parameters which can affect the selectivity and reactor temperature are kept as ‘wish list’ of input variables. At first, all EO reactor operating data (hourly average) of nearly three years were collected. Then after consultation with plant expert, all the input variables that may influence the output variables were noted down. After that, the cross-correlation analysis was carried out. In this method, correlation coefficients of each of the input variables with each output variable and inter input cross-correlation coefficients were found out.

The following criteria are used to shortlist the input variables.
(i) Plant operation experience and knowledge is primarily utilized to identify relevant inputs which can influence catalyst selectivity and reactor temperature.
(ii) For a particular input variable, there should be high cross-correlation coefficient with output variables.
(iii) The values of cross-correlation coefficients of inter input variables should be low.
(iv) The input set of variables were kept as minimum as possible to avoid complexity of the model.

Based on the above criteria 8 input variables are finally shortlisted and tabulated in Table 1.

There are additional two input variables namely inlet EO concentration and inlet moisture concentration which are tackle separately after initial model building.

TABLE 1
Input Output variables for model building and their range

| Variables used in modeling | Data Range |
|---|---|
| Input Variables | |
| Oxygen inlet concentration, mole % (x1) | 2.44-7.84 |
| C2H4 inlet concentration, mole % (x2) | 14.25-35.72 |
| CO2 inlet concentration, mole % (x3) | 0.1-0.64 |
| CG Pressure, bar (x4) | 18.9-23.24 |
| CG Flow (MT/h) (x5) | 659.07-843.98 |
| Work Rate (kg/h m3) (x6) | 72.81-210.59 |
| Cumulative EOE (MT/m3) (x7) | 21.38-4716.21 |
| Total chloride concentration, ppm (x8) | 1.76-7.46 |
| H2O inlet concentration (mol %) (x9) | 0.2-1 |
| EO inlet concentration (mol %) (x10) | 0.001-0.5 |
| C2H6 inlet concentration (mol %) (x11) | 0.05-3 |
| Output Variables | |
| Catalyst selectivity, % (Y1) | |
| Reactor Temperature, ° C., (Y2) | |

“CG” = “cycle gas”

According to one embodiment of the present invention, it has been found that the Input Variables (x4), (x5) and (x7) are-in a relative view-less relevant in the context of the present invention and can thus be omitted, if desired.

The data ranges indicated in the Table 1 for the input variables below are exemplary and may slightly vary between production plants. It has been found that also the inlet concentration of saturated hydrocarbons, in particular the ethane concentration is an advantageous input variable (an example of the feature “process data” as used herein) within the context of the present invention. Preferably, the “total chloride concentration” is the total concentration of all chloride moderators present in the reactor inlet gas stream.

The inventors have found that the above input variables have a different degree of importance for advantageous model building. Thus, according to one aspect of the invention, the inlet variables (an example of the feature “process data” as used herein) used can be grouped as follows:
a) Total inlet chloride moderator concentration
b) Saturated hydrocarbon inlet concentration, in particular ethane inlet concentration
c) CO₂ inlet concentration and/or the oxygen inlet concentration, in particular both the CO₂ and oxygen inlet concentrations
d) Moisture (H₂O) inlet concentration
e) Work Rate
f) C₂H₄ (ethylene) inlet concentration and/or ethylene oxide inlet concentration.

According to a particularly preferred embodiment of the invention, the input variables are a combination of one or more of the aforementioned input variables a) to f), more preferably comprising at least a), more preferably at least a) and b), more preferably at least a) to c), more preferably at least a) to d), more preferably at least a) to e), more preferably a) to f).

### 11. Data Collection, Data Cleaning and Removal of Outliers

In commercial plants, data historian software is used to collect and store every operating parameter at every minute. EO reactor operating data (daily average) for all parameters given in Table 1 from commercial plant of nearly three years were collected. These raw data are then exposed to data cleaning and outlier removal software.

Data cleaning in one of the key tasks of modelling of industrial process as industry data contains noise, spikes, outliers, error etc. due to malfunction of different sensors, transmitters, analyzers, control systems and data historian software etc. It was found that quality of data driven model is determined by quality of data used to define the model. Noisy and bad data can seriously impact the model performance for data driven modelling, data quality is an important factor to be considered.

According to a preferred embodiment of the present invention, an automated effective data cleaning algorithm is part of this invention to avoid manual cleaning. According to this embodiment, multivariate Principal Component Analysis (PCA) was employed for pre-processing of data. An automated MATLAB based program was found which generate a multivariate statistical vector viz. t-squared from the plant operating dataset. Then, in the t-squared vector which have above 95th percentile, the corresponding rows were considered to be outliers and that outliers were removed from the dataset.

### 12. Estimation of Response Time of Various Independent Variables to Affect the EO Catalyst Performance

Selectivity and activity (measured by catalyst temperature) are two performance parameters of EO catalyst. There are 8 process parameters (see Table 2 below) which can affect the selectivity and activity of catalyst. However, in a continuous plant, there is always a delay time of these process parameters to have a visible effect on catalyst performance. For example, if a particular parameter (say CO2 concentration) increases now, we may see the impact on selectivity and activity after 12 hours. This is called time to impact. In short, change in input parameters will have some time delay to see its impact on catalyst performance. Moreover, this delay time or time to impact is different for different process parameters. For some process parameters such as reactor inlet ethylene concentration and oxygen concentration, the impact is immediate i.e., the delay time is very short. On the other hand, reactor inlet CO2 concentration and reactor inlet chloride concentration, the delay time is very large and can be 24 to 48 hours.

In practice, evaluating the response time of individual parameters is a very difficult task as all the parameters are continuously changing in a dynamic plant environment.

In this invention, a statistical methodology is found which can very accurately evaluate the response time of individual parameters from plant past data. Partial correlation co-efficient methodology is applied on EO reactor historical operating data, and the response time is calculated.

Partial correlation is a method used to describe the relationship between two variables whilst taking away the effects of another variable, or several other variables, on this relationship.

Partial correlation co-efficient is a statistical procedure which evaluate the correlation coefficient of an individual process parameters' impact on selectivity or activity while keeping all other variables constant.

Calculations related to partial correlation coefficient:

For simplicity it is assumed that

$$y = f ( {x}_{1} , {x}_{2} )$$

where y is dependent variable and x₁, x₂ are two independent variable. Bivariate (zero order) correlation between y and x1 (r_{yx1}) can be calculated as

$${r}_{y x 1} = \frac{\sum ( {x}_{1} - \overline{{x}_{1}} ) ( y - \overline{y} )}{\sqrt{\sum {( {x}_{1} - \overline{{x}_{1}} )}^{2} \sum {( y - \overline{y} )}^{2}}}$$

where r_{yx1} is bivariate (zero order) correlation between y and x1.

Similarly Bivariate (zero order) correlation between y and x2 (r_{yx2}) can be calculated as

$${r}_{y x 2} = \frac{\sum ( {x}_{2} - \overline{{x}_{2}} ) ( y - \overline{y} )}{\sqrt{\sum {( {x}_{2} - \overline{{x}_{2}} )}^{2} \sum {( y - \overline{y} )}^{2}}} .$$

Bivariate (zero order) correlation between x1 and x2 (rₓ₁ₓ₂) can be calculated as

$${r}_{x 1 x 2} = \frac{\sum ( {x}_{1} - \overline{{x}_{1}} ) ( {x}_{2} - \overline{{x}_{2}} )}{\sqrt{\sum {( {x}_{1} - \overline{{x}_{1}} )}^{2} \sum {( {x}_{2} - \overline{{x}_{2}} )}^{2}}} .$$

These bivariate correlations are used to calculate partial correlations (first order) as follows:

$${r}_{yx 1. x 2} = \frac{{r}_{yx 1} - {r}_{yx 2} {r}_{x 1 x 2}}{\sqrt{( 1 -} {r}_{yx 2}^{2} ) \sqrt{( 1 -} {r}_{x 1 x 2}^{2} )}$$

Where r_{yx1.x2} is the partial correlation between y and x1 while keeping x2 constant. Similarly, partial correlation between y and x2 while keeping x1 constant can be calculated as follows:

$${r}_{yx 2. x 1} = \frac{{r}_{yx 2} - {r}_{yx 1} {r}_{x 1 x 2}}{\sqrt{( 1 -} {r}_{yx 1}^{2} ) \sqrt{( 1 -} {r}_{x 1 x 2}^{2} )} .$$

Multivariate regression equation can be written as y=β₀+β₁x₁+β₂x₂

where β₁=partial slope of the linear relationship between the first independent variable and y β1 indicates the change in y for one unit change in x1, controlling for x2
β₂=partial slope of the linear relationship between the second independent variable and y β2 indicates the change in y for one unit change in x2, controlling for x1
β₀=the y intercept, where the regression line crosses the Y axis.

The partial slopes indicate the effect of each independent variable on y, while controlling for the effect of the other independent variables and can be calculated as follows:

$${β}_{1} = ( \frac{{σ}_{y}}{{σ}_{x 1}} ) [ \frac{{r}_{yx 1} - {r}_{yx 2} {r}_{x 1 x 2}}{1 - {r}_{x 1 x 2}^{2}} ] \\ {β}_{2} = ( \frac{{σ}_{y}}{{σ}_{x 2}} ) [ \frac{{r}_{yx 2} - {r}_{yx 1} {r}_{x 1 x 2}}{1 - {r}_{x 1 x 2}^{2}} ] \\ {β}_{0} = \overline{y} - {β}_{1} \overline{{x}_{1}} - {β}_{2} \overline{{x}_{2}}$$

where σₓ₁ and σₓ₂ are standard deviations of y,x1 and x2 respectively. Once β₁ and β₁ have been calculated, Y intercept β₀ can be calculated by

$${β}_{0} = \overline{y} - β \overline{{1}_{} {x}_{1}} - β \overline{{2}_{} {x}_{2}}$$

The above statistical procedure is used to evaluate the response time of each input variables on selectivity and temperature.

Step 1: Initially time series data are collected from plant for all the input variables in table 1 along with selectivity and temperature and they are arranged with time stamps. Each row of data contains all 10 input variables, selectivity and temperature for a fixed same time stamps. Partial slope of each input parameters are calculated for selectivity and they represent how selectivity varies with each individual parameter while keeping all other input constant.

It is very crucial to evaluate the individual input parameters contribution to overall selectivity and temperature. In dynamic plant environment this type of data are not available where only one input variable varies and all other input parameters remain constant.

Step 2: Now all input data are rearranged so that all input parameters at time stamp (t−1) is aligned with selectivity at time stamp t in a single row.

In other words, individual parameters are arranged in such a way that current selectivity is aligned with data of previous one hr data of input parameters. After all the time series data are shifted and realigned in above way, partial slope of each input parameters are calculated for selectivity in similar way.

Step 3: step 2 is repeated for 47 times and each time partial slope of each parameter are calculated with data shifted 2 hr, 3 hr . . . 48 hr in past. In other words, individual parameters are arranged in such a way that current selectivity is aligned with data of (t−2), (t−3) . . . (t−24) hr data of input parameters.

Partial slopes of each variable in table 1 are evaluated for all these cases.

Step 4: A table is formed which tabulate all the partial slopes of each individual parameter with selectivity for time t, (t−1), (t−2) . . . (t−48) where t=t represents current time, (t−1) represents previous hour and so on. Out of these 49 partial slopes for each individual parameter, the time when this partial slopes comes maximum, it is considered the response time of the partial input parameters. For example, say for input parameters x1, partial slope of (t−8) comes highest (ignoring plus or minus sign of slope), then 8 hr is considered as delay time for variable x1 on selectivity. Same process is repeated for all other parameters.

Step 5: step 1-4 repeated separately for reactor temperature also Table 2 summarizes the results of the above calculations.

TABLE 2
Response time of input variables for selectivity and temperature

| | Delay time (in hrs) | |
|---|---|---|
| | Catalyst | Reactor |
| Input Variables | Selectivity | temperature |
| Oxygen inlet concentration, mole % (x1) | 1 | 3 |
| C2H4 inlet concentration, mole % (x2) | 4 | 5 |
| CO2 inlet concentration, mole % (x3) | 7 | 5 |
| CG Pressure, bar (x4) | 10 | 10 |
| CG Flow (MT/h) (x5) | 2 | 2 |
| Work Rate (kg/h m3) (x6) | 26 | 23 |
| Cumulative EOE (MT/m3) (x7) | 28 | 24 |
| Total chloride concentration, ppm (x8) | 9 | 3 |

It is worth mentioning here that these delay times are not universal for any EO reactor, but they are dependent on catalyst type, plant size, catalyst age etc. However, this procedure to calculate delay time from actual plant data is generic and can be applied to any EO reactor plant data to evaluate delay time for that particular plant.

Accurate estimation of response time for individual process parameters as set out above is a very preferred embodiment of the present invention and allow an important improvement in quantitatively calculating the future value of performance parameters. This will help to manipulate the current value of controlled parameters i.e., to take corrective action so that performance parameters do not deteriorate in future. This also helps to take accurate calibrated corrective actions to improve the performance parameters in future.

### 13. Modelling Through Multi Gene Genetic Programming

Once the delay times of each parameter are calculated, input and output data set are shifted by their respective delay time and realigned. This realignment of data helps to predict selectivity and temperature with more accuracy. The cleaned realigned data was taken for MGP-based modelling. Random partitioning of the dataset comprising of eight input variables and two output variables (Table 1) into training set (80% of whole data) and test set (20% of whole data) was performed. The training set was used to compute the model by maximizing the fitness value whereas the test data set was used for cross-validation of the expression developed. The main objective of the cross-validation is to make the model more generalizable.

Code written in MATLAB 2019a was used to find the MGP-based model. In this invention, root-mean-squared error (RMSE) between actual and predicted outputs was taken as fitness function and the program was run in such a manner that the value of RMSE got minimized. Due to the stochastic nature of the MGP, the program was run 100 times to find the model.

### Results And Discussions

### 14. Performance of GP Model

Main objective of present invention is to generate a closed for model equation of EO reactor which is accurate, simple, portable and explainable.

The three years hourly average dataset were subjected to cleaning and after removal of outliers, 16000 data sets were qualified for model building. The values of MGP parameters required for modelling were found based on trial-and-error approach. For a real world reactor, the present invention has exemplary been verified in that basic arithmetic operators and functions, population size of 250, maximum generation of 500, maximum tree depth of 4 and maximum number of genes of 6 were taken for modelling. If these parameters are taken with high value, the model accuracy may increase but the complexity of the solutions also increases and also the program becomes computationally expensive.

15. Finding Closed Form Model Equations

MGP generates lot of promising model equations during its run. Table 2 and 3 summarizes the most prominent closed form equations generated by GP for selectivity and temperature. As seen in Table 2 and 3, there is always conflicting objective between model complexity (denoted by number of tree nodes) and model prediction accuracy (denoted by R2 in table). More accurate models are more complex and vice versa. Complex models are difficult to interpret and unnecessary over fit the data. For each run of MGP, two Pareto diagrams were found, one is for catalyst selectivity and another is for reactor temperature (FIGS. 6(a) and 6(b)). Pareto diagrams represent the plot of expressional complexity (represented by number of nodes in the equation) vs. prediction accuracy represented by R2. Optimum model is determined from the available models by choosing the model with very high value of R2 and also reasonably low complexity. The red triangular points in Pareto diagrams were chosen as examples.

### 16. Controlling Model Complexity

Use of multigene regression models frequently suffers from the phenomenon called ‘bloat’, vertical and horizontal bloat. Vertical bloat refers the tendency to evolve trees that contain terms that confer little or no performance benefit. In terms of model development, this is related to the phenomenon of overfitting. In present invention, by restriction on tree depth and use of Pareto tournament between expressional complexity and accuracy the vertical bloats were ameliorated. Tree depth was kept 4 by trial-and-error method and pareto diagrams are generated as shown in FIGS. 6(a) and 6(b).

Horizontal bloat is the tendency of multigene models to acquire genes that are either performance neutral (i.e., deliver no improvement in R2 (or “R2” as also used herein) on the training data) or offer very small incremental performance improvements. Horizontal bloat is essentially the same behavior exhibited by non-regularized models, where it is well known that the addition of model terms leads to a monotonically increasing R2 on training data even though the terms may not be meaningful (e.g., they are capturing noise) or allow the model to generalize well to testing or validation data sets. Ostensibly, the simplest way to prevent horizontal bloat in multigene regression is to limit the maximum allowed number of genes in a model. In this invention, maximum number of genes are kept 6 after trial-and-error method.

### 17. Shortlisting the Models

Out of potential candidates or representative model equations (refer Table 3 and 4) with varying degree of complexity and accuracy, for selection of optimum model, the following criteria were kept in mind:
(i) Simplicity: The model complexity should be as low as possible.
(ii) Prediction accuracy: The developed model should have low RMSE and high R2
(iii) The model equation should capture the underlying physics of the process. In other words, model equations not merely a predictive correlation but also should have physical sense of the system under invention. This is a prime consideration to develop real life reactor models. To judge this capability, domain expert's qualitative knowledge about the reactor behavior is collected from plant and technology licensor. Their plant operating knowledge, experience and observations of reactor behavior are summarized in Table 4.

TABLE 2

| Model | | Model | |
|---|---|---|---|
| ID | R2 | complexity | Model |
| 23 | 0.967 | 122 | 0.658 x1 + 0.00139 x2 − 24.2 x3 − 0.667 x4 + 0.00416 x5 − |
| | | | 0.00139 x6 − 0.00139 x7 + (0.658 x51/2)/x7 − 1.19e−4 x62 + |
| | | | 0.00139 x82 + 0.00589 x32 x4 x6 + (2.6e−4 x82 (1.01 x3 + |
| | | | x5 − 1.0 x7))/(x2 x3) + 106.0 |
| 51 | 0.967 | 92 | 0.655 x1 − 24.1 x3 − 0.665 x4 + 0.00418 x5 − 0.00139 x7 + |
| | | | (0.655 x51/2)/x7 − 1.22e−4 x62 + 0.00586 x32 x4 x6 − (2.56e−4 |
| | | | x82 (x7 − 1.0 x5 + x32))/(x2 x3) + 106.0 |
| 75 | 0.967 | 110 | 0.658 x1 − 24.1 x3 − 0.667 x4 + 0.00418 x5 − 0.00139 x6 − |
| | | | 0.00139 x7 + (0.658 x51/2)/x7 − 1.18e−4 x62 + 0.00587 x32 |
| | | | x4 x6 + (2.56e−4 x82 (1.06 x3 + x5 − 1.0 x7))/(x2 x3) + 106.0 |
| 97 | 0.967 | 118 | 0.658 x1 − 24.1 x3 − 0.668 x4 + 0.00418 x5 − 0.00139 x6 − |
| | | | 0.00139 x7 + 0.00139 x8 + (0.658 x51/2)/x7 − 1.18e−4 x62 + |
| | | | 0.00587 x32 x4 x6 + (2.56e−4 x82 (1.06 x3 + x5 − 1.0 x7))/(x2 |
| | | | x3) + 106.0 |
| 200 | 0.967 | 108 | 0.655 x1 − 24.1 x3 − 0.665 x4 + 0.00418 x5 − 0.00139 x7 + |
| | | | (0.655 x51/2)/x7 − 1.22e−4 x62 + 0.00586 x32 x4 x6 + |
| | | | (2.56e−4 x82 (1.06 x3 + x5 − 1.0 x7))/(x2 x3) + 106.0 |
| 255 | 0.936 | 46 | 0.336 x1 − 0.00168 x2 − 26.8 x3 − 0.00168 x7 − 0.338 x8 + |
| | | | 0.259 x32 (x2 + x8 + x71/2) + 95.4 |
| 260 | 0.562 | 8 | 1.39e−5 x62 x8 − 29.2 x3 + 94.1 |
| 293 | 0.926 | 32 | 0.265 x1 − 17.1 x3 − 0.00144 x7 − 0.265 x8 + 0.0361 x32 |
| | | | (x6 + x71/2) + 93.4 |
| 303 | 0.956 | 49 | 0.431 x1 − 18.8 x3 − 0.00186 x7 − 0.42 x8 + 0.112 x32 (x3 + |
| | | | x6) − 5.7e−5 x42 x6 + 0.0107 x71/2 + 97.3 |
| 304 | 0.584 | 9 | 75.4 x3 − 114.0 x31/2 + 126.0 |
| 316 | 0.958 | 50 | 0.449 x1 − 20.8 x3 − 0.00184 x7 − 0.449 x8 − 5.39e−5 x42 |
| | | | x6 + 0.101 x32 (x6 + x71/2) + 97.7 |
| 514 | 0.916 | 30 | 0.00214 x5 − 0.0307 x6 − 0.00214 x7 − 0.00214 x8 + 94.5 |
| 571 | 0.89 | 23 | 0.0168 x3 − 0.0168 x5 − 0.00184 x7 + 0.0187 x34 + 103.0 |

TABLE 3

| Model | | Model | |
|---|---|---|---|
| ID | R2 | complexity | Model |
| 9 | 0.988 | 178 | 0.816 x3 − 0.635 x1 + 0.272 x4 + 0.272 x6 + 0.272 x8 + |
| | | | (3.55e−15 (1.62e+14 x2 + 1.08e+14 x6 + 5.41e+13 x7))/ |
| | | | (x4 + x6)1/2 − 1.18 (2.0 x6 + x7)1/2 − 2.19e−4 x7 (x1 + x71/2) + |
| | | | 0.0017 (x3 + x6)1/2 (x7 + x12 + x42) − 4.83e−4 x42 x6 + |
| | | | 223.0 |
| 55 | 0.988 | 182 | 0.807 x3 − 0.734 x1 + 0.269 x4 + 0.269 x6 + 0.269 x8 − 1.27 |
| | | | (2.0 x6 + x7)1/2 − 2.29e−4 x7 (x1 + x71/2) + 0.00178 (x3 + |
| | | | x6)1/2 (x7 + x12 + x42) − 4.65e−4 x42 x6 + (4.44e−16 |
| | | | (1.37e+15 x2 + 4.58e+14 x6 + 4.58e+14 x7 + 4.58e+14 |
| | | | x12))/(x4 + x6)1/2 + 225.0 |
| 61 | 0.988 | 174 | 0.775 x3 − 0.657 x1 + 0.258 x4 + 0.258 x6 + 0.258 x8 − 1.17 |
| | | | (2.0 x6 + x7)1/2 − 2.22e−4 x7 (x1 + x71/2) + 0.00172 (x3 + |
| | | | x6)1/2 (x7 + x12 + x42) − 4.36e−4 x42 x6 + (1.78e−15 |
| | | | (5.43e+13 x2 + 5.43e+13 x6 + 1.09e+14 x7 + 3.43e+13 |
| | | | x12))/(x4 + x6)1/2 + 225.0 |
| 254 | 0.988 | 91 | 5.91e−4 (x4 + x7 + x31/2)2 − 4.34e−4 (2.0 x4 + x7)2 − 1.84e−4 |
| | | | (x6 + x7 + x8)2 − 1.77e−4 (x1 + x7 + x71/2)2 + 1.99e−4 |
| | | | (x6 + x7 + x8 + 2.0 x71/2)2 + 227.0 |
| 258 | 0.955 | 27 | 2.07e−5 (x6 + x7 + x8)2 − 2.04e−5 (2.0 x4 + x7)2 + 228.0 |
| 286 | 0.987 | 86 | 4.04e−4 (x6 + x7 + x8 + x8/x6 + x71/2)2 − 2.71e−4 (x4 + |
| | | | x7 + x31/2)2 − 1.71e−4 (x1 + x7 + x71/2)2 − 3.87e−4 (x6 + x7 + |
| | | | x8)2 + 4.22e−4 x72 + 227.0 |
| 291 | 0.976 | 48 | 1.84e−5 (x6 + x7 + x8 + 2.0 x71/2)2 − 1.88e−5 (x1 + x7 + |
| | | | x71/2)2 + 226.0 |
| 321 | 0.987 | 87 | 9.83e−5 (x4 + 2.0 x7)2 − 1.86e−4 (x6 + x7 + x8)2 − 2.4e−4 |
| | | | (2.0 x4 + x7)2 − 1.73e−4 (x1 + x7 + x71/2)2 + 2.03e−4 (x6 + |
| | | | x7 + x8 + 2.0 x71/2)2 + 227.0 |
| 326 | 0.988 | 95 | 5.81e−4 (x4 + x7 + x31/2)2 − 4.24e−4 (2.0 x4 + x7)2 − 3.83e−4 |
| | | | (x6 + x7 + x8)2 − 1.76e−4 (x1 + x7 + x71/2)2 + 3.98e−4 |
| | | | (x6 + x7 + x8 + x8/x6 + x71/2)2 + 227.0 |
| 327 | 0.988 | 93 | 5.81e−4 (x4 + x7 + x31/2)2 − 4.24e−4 (2.0 x4 + x7)2 − 3.83e−4 |
| | | | (x6 + x7 + x8)2 − 1.76e−4 (x1 + x7 + x71/2)2 + 3.98e−4 |
| | | | (x6 + x7 + x8 + x8/x6 + x71/2)2 + 227.0 |
| 549 | 0.97 | 34 | 0.107 x8 − 1.28 x4 − 0.107 x2 + 0.0414 (x6 x7)1/2 + (5.52 |
| | | | x1 x8)/(x8 + x12) + 243.0 |
| 554 | 0.949 | 9 | 0.0413 (x6 x7)1/2 + 218.0 |
| 555 | 0.986 | 65 | 0.565 x8 − 2.65 x4 − 1111.0/(x11/2 x7) − 24.9 (x6 x7)1/2 + |
| | | | 25.0(x4 + x6 + x6 x7)1/2 + (1.64 x1 x4)/(x8 + x12) + 259.0 |
| 559 | 0.981 | 61 | 12.4 (x4 − 1.0 x2 + x6 + x6 x7)1/2 − 2.97 x4 − (0.352 x4)/ |
| | | | x8 − 12.4 (x6 x7)1/2 − 0.0121 (x6 + x7)1/2 + 281.0 |

TABLE 4

| Sl. | Parameters changed keeping all | What happen | What happen to |
|---|---|---|---|
| no | other parameters constant | to selectivity? | temperature? |
| 1 | If oxygen inlet | Increase | Decrease |
| | concentration increase | | |
| 2 | If ethylene inlet | Increase | Decrease |
| | concentration increase | | |
| 3 | If CO₂ inlet | Decrease | Increase |
| | concentration increase | | |
| 4 | If CG Pressure increase | Increase | Decrease |
| 5 | If CG flow increase | Increase | Decrease |
| 6 | If Work rate increase | Decrease | Increase |
| 7 | If Cum EOE/m³ of | Decrease | Increase |
| | catalyst increase | | |

All the ten equations in Table 2 and 3 are subjected to the above scrutiny to judge whether the developed equation is in agreement with plant observations. All the developed models are passed through rigorous testing. For example, ten test data set was generated where all the variables are kept at their 50-percentile value except oxygen inlet concentration which was varied from its minimum value to maximum value by equal 10 intervals. These test data were put in equations of Table 2 and Table 3 and respective 10 set selectivity and temperature data were generated. After that, first oxygen inlet concentration vs selectivity and oxygen inlet concentration vs temperature were plotted. From these plots, observation number 1 of Table 2 was verified. In same way all other plots were generated which are shown in FIGS. 7 and 8 for selectivity and temperature.

Model equations that do not follow the observations in Table 4 are rejected, as they do not capture the underlying physics of the EO reactor and not in agreement with plant observations. They merely represent a complex data fitting equation without actual sense.

From the shortlisted model equations, only one model equation for catalyst selectivity and one model for reactor temperature were finally selected which have been mentioned as below (Equation 8 and Equation 9): These two equations are considered the representative model equation for selectivity and temperature as they are highly accurate, obey the Table 4 observations and capture the internal physics of the underlying exemplary reactor.

$${Y}_{1} = 0.658 {x}_{1} + {0.00138}_{2} - 24.2 {x}_{3} - 0.667 {x}_{4} + 0.00416 {x}_{5} - 0.00139 {x}_{6} - 0.0039 {x}_{7} + ( 2.64 \times {10}^{- 4} {x}_{\text{?}}^{2} ) / {x}_{2} + ( 0.658 {x}_{\text{?}}^{1 / 2} ) / {x}_{7} - 1.19 \times {10}^{- 4} {x}_{\text{?}}^{2} + 0.00139 {x}_{8}^{2} + 0.00589 {x}_{\text{?}}^{2} {x}_{\text{?}} {x}_{6} + ( 2.64 \times {10}^{- 4} {x}_{\text{?}} {x}_{\text{?}}^{2} ) / ( {x}_{2} {x}_{\text{?}} ) - ( 2.64 \times {10}^{- 4} {x}_{7} {x}_{8}^{2} ) / ( {x}_{2} {x}_{\text{?}} ) + 106. \qquad ( 8 )$$

$${Y}_{2} = 0.807 {x}_{3} - 0.734 {x}_{\text{?}} + 0.269 {x}_{4} + 0.269 {x}_{6} + 0.269 {x}_{8} - 1.27 {( 2. {x}_{6} + {x}_{7} )}^{1 / 2} - 2.29 \times {10}^{- 4} {x}_{7} ( {x}_{1} + {x}_{7}^{1 / 2} ) + 0.00178 {( {x}_{\text{?}} + {x}_{6} )}^{1 / 2} ( {x}_{7} + {x}_{1}^{2} + {x}_{4}^{2} ) - ( 4.44 \times {10}^{- 16} ( 1.37 \times {10}^{- 16} {x}_{2} + 4.58 \times {10}^{\text{?}} {x}_{6} + 4.58 \times {10}^{\text{?}} {x}_{7} + 4.58 \times {10}^{14} {x}_{1}^{2} ) ) / {( {x}_{4} + {x}_{6} )}^{1 / 2} + 223. \qquad ( 9 )$$

$$\text{?} \text{indicates text missing or illegible when filed}$$

The corresponding Coefficient of Determination (R2) and Average Percentage Error (APE) of the above models for training and test data are reflected in Table 5.

TABLE 5

| | Training | | | Testing | | |
|---|---|---|---|---|---|---|
| Model | R² | APE | RMSE | R² | APE | RMSE |
| Catalyst selectivity | 0.967 | 0.158 | 0.207 | 0.974 | 0.408 | 0.194 |
| model | | | | | | |
| Reactor temperature | 0.988 | 0.272 | 0.366 | 0.982 | 0.315 | 0.459 |
| model | | | | | | |

Looking at high values of R2 and low values of APE for both the models involving catalyst selectivity and reactor temperature (Table 5), it can be concluded that the predicted output values are at par with the actual output values and the models developed are reliable, fairly accurate and captures the inherent physics of EO reactor. The high R2 value on unseen test data and low APE also indicates about the model's generalizability and accurate learning on nonlinear input and output relationship.

Models' prediction performance on training and testing data are shown in FIG. 9. Almost overlapping nature of actual vs predicted curve indicates model's good prediction accuracy.

From Table 5 and FIG. 9 it is concluded that developed model is highly accurate and reliable as it also performs well with unseen test data.

### 18. Generation of Explainable Model Equations

One of the major advantages of MGP modelling technique over ANN and SVR techniques is that it generates closed form of equation (like equation 8 and 9), which is explainable, portable and easily implantable in plant distributed control system. Though MGP generates a closed form of equation which has very high predictive capability, the equation found is large and complex and sometimes difficult to directly interpret. In present invention, a methodology is found to enhance the interpretability of the developed equations. FIG. 6 and FIG. 7 summarize the found methodology. Those figures are generated by changing one variable at a time from its minimum to maximum value (10 steps) while keeping all other 7 input variables at their 50-percentile value. Selectivity equations developed by MGP is used to predict the selectivity value in each case of these simulated test data. After plotting was done, a trend line was drawn through each data whose equation and R2 value is shown in figure. Based on visual inspection and R2 value trend line curve was selected (like straight line, or polynomial with degree 2 or 3 or more) so that generated trend line almost matches with the data. As seen from the FIG. 6 and FIG. 7, the trend lines are very decisive and monotonically increasing and decreasing. As mentioned earlier, they all match the plant actual observations and obey the Table 4.

In short, found models capture the nonlinear relationship between selectivity and reactor operating parameters. These trend lines can be used by plant operating engineers to get the insights how a particular input parameter affects the catalyst selectivity. For example, from FIG. 6, it is quantitively clear that increasing inlet oxygen and ethylene concentration, actually enhance the catalyst selectivity whereas increasing CO2 reduces the selectivity.

The selectivity increases linearly with oxygen concentration with positive slope of 0.658, whereas the relation of selectivity with ethylene and CO2 are nonlinear and represented by second order polynomial. Now these trend line equations have been considered when finding the following equations (Equation 10 and 11).

$$Selectivity = 0.658 ( {x}_{1} - {x}_{1 , avg} ) + [ - 0.0013 ( {x}_{2}^{2} - {x}_{2 , avg}^{2} ) + 0.0993 ( {x}_{2} - {x}_{2 , avg} ) ] + [ 13.95 ( {x}_{3}^{2} - {x}_{3 , avg}^{2} ) - 16.962 ( {x}_{3} - {x}_{3 , avg} ) ] - 0.5572 ( {x}_{4} - {x}_{4 , avg} ) + 0.0045 ( {x}_{5} - {x}_{5 , avg} ) + [ - 0.0001 ( {x}_{6}^{2} - {x}_{6 , avg}^{2} ) + 0.0136 ( {x}_{6} - {x}_{6 , avg} ) - 0.0018 ( {x}_{7} - {x}_{7 , avg} ) + [ - 0.0448 ( {x}_{8}^{2} - {x}_{8 , avg}^{2} ) + {10}^{- 12} ( {x}_{8} - {x}_{8 , avg} ) ] + 84.98 \qquad ( 10 )$$

$$Temperature = 0.8435 ( {x}_{1} - {x}_{1 , avg} ) + 0.0446 ( {x}_{2} - {x}_{2 , avg} ) + 1.0246 ( {x}_{3} - {x}_{3 , avg} ) - 2.5209 ( {x}_{4} - {x}_{4 , avg} ) + [ 0.0001 ( {x}_{6}^{2} - {x}_{6 , avg}^{2} ) + 0.0808 ( {x}_{6} - {x}_{6 , avg} ) ] + 0.0066 ( {x}_{7} - {x}_{7 , avg} ) + 0.269 ( {x}_{8} - {x}_{8 , avg} ) + 222. \qquad ( 11 )$$

Where x1, x2, . . . x8 are the actual value of the 8 input variables and x1, avg, x2, avg, . . . x8, avg are the average (50 percentile) value of input variables respectively.

Each term in the equation 10 represents the change in selectivity if a particular parameter deviates from its average value. For example, the term, (x1-x1.) represents the deviation of inlet oxygen concentration from its average value and when it multiplied by co-efficient 0.658, represents the selectivity gain (or penalty) due to oxygen. In this way, all 8 parameters contribution is calculated in equation 8 and it is added with about 88.5% (average selectivity) to get the actual selectivity.

Main advantage of this equation (equation 10) over GP equation (equation 8) is that this equation is interpretable and easily explainable to plant engineer. Equation is simple, and contains terms or parametric co-efficient which throw lights relative importance of each parameter on the overall selectivity if they deviate from these base values. Also, it indicates whether the effect of each parameters is linear or non-linear.

Equation 10 is then used to predict selectivity of three-year actual data and predicted and actual selectivity is compared. Prediction error is 0.8% and R2 is 0.97. This low value of prediction error and high value of R2 signifies that developed equation (equation 10) is highly accurate and reliable.

### 19. Estimation of Parametric Coefficients with Aging of Catalyst

Selectivity and activity are two major performance parameters of EO catalyst. There are input parameters which have an influence on selectivity and activity. Parametric coefficients are defined as the change in selectivity or activity when one unit change of any individual parameter is done. For example, oxygen inlet concentration parametric coefficient of selectivity is defined as:

$$Oxygen Parametric coefficient \frac{Change in selectivity}{Unit change in {O}_{2} inlet concentration}$$

### Usage of Parametric Coefficient

Parametric coefficients are very important to calculate the change in selectivity or activity of catalyst when an input parameter changes. It also provides a calculation basis to calculate the adjusted selectivity and activity to judge the real performance of catalyst in midst of ever changing dynamic plant environment.

At present, the value of parametric coefficient of limited number of parameters are available for Syndox-400 catalyst. These values are estimated in controlled laboratory environment and not in the plant environment. The validity of these parametric coefficients in plant environment is questionable as they were built in controlled R&D environment. It is very crucial to estimate the real life parametric co-efficient to enable them to apply in real time optimization.

### Estimations of Parametric Co-Efficient

As a part of the present invention, a calculation methodology is found to estimate the parametric coefficient of different input variables from actual plant data. The above mentioned Genetic programming based modelling methodology is used to calculate the parametric co-efficient of each parameters as expressed in equation 10 and 11. The whole 3 years plant hourly data is utilized to arrive at equation 10 and 11.

Parametric co-efficient of each individual inputs are derived from equation 10 and 11 and summarized in table 6:

TABLE 6

| | Parametric co-efficient | |
|---|---|---|
| | Catalyst | Reactor |
| Input Variables | Selectivity | temperature |
| Oxygen inlet concentration, | 0.658 | 0.8435 |
| mole % (x1) | | |
| C2H4 inlet concentration, | [−0.0013, 0.0993] | 0.0446 |
| mole % (x2) | | |
| CO2 inlet concentration, | [13.95, −16.962] | 1.0246 |
| mole % (x3) | | |
| CG Pressure, bar (x4) | −0.5572 | −2.5209 |
| CG Flow (MT/h) (x5) | 0.0045 | 0 |
| Work Rate (kg/h m3) (x6) | [−0.0001, 0.0136] | [0.0001, 0.0808] |
| Cumulative EOE (MT/m3) (x7) | −0.0018 | 0.0066 |
| Total chloride concentration, | [−0.0448, 10-12] | 0.269 |
| ppm (x8) | | |

### Estimations of Parametric Co-Efficient at Different Age of Catalyst

The value of parametric coefficients changes with age of catalyst. The sensitivity at different input parameters on catalyst selectivity and activity changes in a non-linear fashion over the age of catalyst. The current set of available parametric coefficients does not take this in account. It is beneficial to estimate the parametric co-efficient at different age of catalyst.

Whole set of 3 years' data are divided in 5 sets with 6 months each. Same modelling methodology is applied to each 5 sets data separately and parametric co-efficient are evaluated each time. In other words, same calculations are repeated in different catalyst age at fixed time intervals, say for every 6 months and age sensitivity coefficient of each parameter are calculated from historical data.

In this way, parametric co-efficient at different age of catalyst can be estimated.

### Parametric Coefficient of Inlet Moisture and inlet EO

There are few instances in plant when inlet moisture and/or inlet EO has increased significantly in plant due to some disturbances in downstream plant or during summer time etc. It is well known that increase of inlet moisture and/or inlet EO can have a very detrimental effect on catalyst selectivity and activity.

Parametric co-efficient of inlet EO and inlet moisture are separately calculated due to the fact the data related to very high inlet EO and inlet moisture are very sparse and few.

Following calculations procedures are followed:
1. Data related to high inlet moisture and high inlet EO are extracted from whole data sets and keep in a separate database.
2. These new database is then used to evaluate the effect of moisture and EO on catalyst selectivity.
3. Selectivity is expressed e.g. by equation 10. Two additional terms for inlet EO and inlet moisture are added in similar way in equation 10. Two unknown additional parametric co-efficient is now calculated by regression analysis which give minimum error with new data sets.
4. Equation 10 along are converted to a simple explainable form as given below with two additional terms for moisture and inlet EO can be expressed as

$$S = \sum {a}_{i} ( {x}_{i} - {x}_{i , avg} ) + {C}_{1}$$

5. Purpose to do separate calculations due to the fact that very low volume of data is available containing high inlet moisture and high inlet EO. If earlier methods are followed these low volume data get lost amid large volume of normal datasets.

The parametric coefficient of inlet moisture, inlet EO are not available for Syndox-400 catalyst and this procedure gives a methodology to evaluate them from real life plant data.
When these coefficients are used in the above equation, it predicts the selectivity and catalyst temperature accurately.
Average prediction error—0.44%, R2-0.99 for a commercial EO plant.

### Module for Identification of Current Status of EO Reaction Process

For an online digital twin application, it is very crucial to understand where the process stands currently in terms of catalyst performance. This will subsequently help to determine the preventive and corrective actions.

Catalyst performance status is detected online by 3 key performance indexes (KPI) namely adjusted selectivity, adjusted temperature and calculated EOE production.

In real plants process parameters and above 3 KPIs have noise coming from various measuring transmitters. To avoid influence of noise for online automated decision making following calculations are followed:

Validated selectivity, temperature and EOE data are taken for each hour and for last 24 hours.

A trend line of cubic polynomials is automatically calculated and drawn to smoothen the noisy data. A pipeline was drawn around this trend line with radius of one standard deviations. The smooth trend lines are calculated and drawn for each parameters and KPIs around reactor.

These smooth lines are then taken for decision making. A decision making truth table related to selectivity, temperature and production rate based on first principle can be prepared to decide on current chloride process status.

### 21. Online Module for Root Cause Analysis in EO Reactor

During operation of EO catalyst in actual plant, there are times when catalyst performance (selectivity and/or activity) gets deteriorated in spite of full effort of engineers and operators running the plant. Production engineer are very curious to know why this performance deterioration happened.

This online module will identify all the responsible parameters for selectivity and/or activity decline in every hour. It will help to identify the root cause for selectivity and activity drop. All responsible parameters will be shown in a comprehensive infographic

[symbol]

The contribution of each parameter on selectivity and activity trend will be calculated based on parametric coefficients of table 3. A replica of GP EO reactor model will run in the background to evaluate individual contributions. Chloride contribution to selectivity and activity will be calculated as remaining delta after allocating contributions with other parameters
5) Root cause analysis of downstream of plant (outside EO reactor) which affects catalyst performance

GP based model are also built similarly for the following two systems:
Cycle water system comprising EO scrubber and EO stripper
CO2 removal system comprising CO2 absorber and regenerator.

This online module will Identify root cause outside EO reactor area, if any with the help of above two models. This will scan process parameters in EO scrubber and EO stripper, wash tower, CO2 absorber and regenerator etc. and identify root cause for high EO, CO2 and Moisture at reactor inlet. Based on Root Cause Analysis it provides recommendation for remedial action in real time basis.

### Current Chloride Zone Detection

Few ppm chloride is added at cycle gas at reactor inlet to improve catalyst selectivity and activity. For SynDox 400 series high selectivity catalyst, selectivity and activity follows a curve as shown in figure. As seen from the inverted U shaped curve, there is an optimum chloride value which gives highest selectivity. Selectivity drops significantly if the chloride level is below or above this optimum value. It is very crucial to identify and run the catalyst at optimum chloride level.

### Exemplary Achievement of the Present Invention use as a Real Time Optimizer in EO Reaction System to Detect Optimum Chloride Level

EO reactor use silver based catalyst and Ethylene Glycol plant economics greatly depends on catalyst selectivity and activity.
Catalyst's selectivity and activity is very sensitive to chloride and other process parameters
A very minor change in chloride can reduce the catalyst selectivity drastically
Plant engineers could not always detect the optimum chloride in real time basis and thus loses 0.5-1% selectivity on an average throughout the catalyst life. This leads to erosion of potential productivity and profits in terms of million USD per year for a moderate size glycol plant.
Due to the above reasons, full potential of catalyst performance could not be realized in actual plant.
This resulted in huge monetary loss of plant operators.

### Major Challenges for Chloride Optimization in Ethylene Oxide Reactor

Optimum chloride concentration changes with time, temperature, age of the catalyst which are difficult to calculate as no credible phenomenological chloride model is available.
Different process parameters like ethylene concentration, ethane concentration, temperature etc. can change the chloride inventory on catalyst surface and shift the optimum chloride point in dynamic plant environment
Catalyst selectivity and activity is determined by the chloride concentration on catalyst surface (which has no measurement).
The erroneous reading of chloride analyzers and mass spec analyzers makes the situation difficult to follow and interpret in real plant

### Challenges Engineers are Faced with when Optimizing Chloride on Real Time Basis

Chloride optimization needs deeper understanding of chloride impact on catalyst. A novice chemical engineer/operator face difficulty to have holistic understanding.
Most of the operating plants bring fresh engineers and operators periodically and they took 1-2 years to understand the chloride phenomena.
The faulty reading of mass spec, flow, chloride analyzers around the EO reactors. mislead the operators for chloride optimization
The delayed effect (8-48 hours) of chloride and continuous and random disturbance in dynamic plant environment make it difficult to properly follow and interpret the chloride impact in real time.

### How Current Chloride Zone is Detected by Means of the Present Invention

The decision of current chloride (chlorination) state detection can be based on the chloride curve as shown e.g. in FIG. 10. It is a complex decision making process and holistic decision need to be taken by evaluating chloride change and its impact on adjusted selectivity, adjusted temperature and calculated EOE. Following decision making algorithm is developed to detect current chloride zone in real time:
1. Change in EDC flow is calculated for past and exactly one delay time before for chloride as given in delay time table from time series data of actual plant.
2. Change in adjusted selectivity and adjusted temperature is calculated for last one hour from time series data. Equation 10 and 11 are used to calculate the above two respectively.
3. Adjusted selectivity and adjusted temperature is considered here for decision making instead of actual selectivity and temperature to negate all the effects of other parameters on selectivity. Change in adjusted selectivity and adjusted temperature represents the true selectivity and temperature change due to chloride only. This is very crucial to detect change in true selectivity and temperature due to chloride only to make an accurate decision.
4. For ease of decision making, the selectivity and chloride curve is divided in five zone as shown in FIG. 10. Green curve depicts the selectivity with chloride for a typical Syndox 400 series high selectivity catalyst.
   Zone I: Severe under chloride zone
   Zone II: Mild under chloride zone
   Zone III: Optimum chloride zone
   Zone IV: Mild over chloride zone
   Zone V: Severe over chloride zone
      5. Based on e.g. last 8 hrs adjusted selectivity, adjusted temperature and calculated EOE, algorithm may determine the current chloride status in real time and decide on corrective and preventive actions.

### 22. Prediction Module

Based on the current status of the plant, future selectivity, temperature and EOE will be predicted for e.g. next 24 hours with this hybrid model in real time basis. The artificial intelligence based GP model will be used to predict the selectivity.

This GP model has superior prediction capability and explain ability. It will capture different parametric co-efficient at different age.

This future prediction capability of digital twin helps the plant engineer to see e.g. 24 hrs or even farther ahead in future due to their past action. Any future selectivity, temperature and EOE production deterioration trend is shown in dashboard with infographics in real time basis. These future predictions are recalculated at fixed or variable time intervals, e.g. every hour based on plant current data and the trends are updated.

### 23. Optimization Module

Purpose of optimization module is to analyze the current situations of the plant, evaluate the best move of manipulated variables to maximize catalyst selectivity and activity. Based on GP model prediction an online metaheuristic optimization (multi objective genetic algorithm) is carried out to prescribe operators the best operating condition in current situation to achieve the best selectivity and temperature. This metaheuristic optimization will explore thousands of possible combinations of corrective and preventive action that can be taken and prescribe the best among them to the operators. During calculations of best move, optimization modules need to honor the different constraints of process parameters, which is given by plant engineers. This optimization module exploits any margins available in ethylene and oxygen concentrations, cycle gas pressure and flow, EDC flow etc. which can be changed/manipulated by plant engineers. This online optimizer will guide and recommend the optimum process parameters which need to be maintained in the plant to the panel operators. The effect of optimization will be shown in dashboard and operators will clearly see what benefits of selectivity and activity plant is going to get in future if he implements the recommended actions.

### 24. Optimization Calculations Through Multi-Objective Genetic Algorithm (MOGA)

Once a reliable model is developed, the second objective of present invention is to utilize the developed model to increase profit of the ethylene oxide plant and simultaneously reduce its environmental impact. This done by applying nature inspired metaheuristic optimization algorithm on the model to optimize the input process parameters which simultaneously maximize selectivity (i.e. reactor performance) and minimize reactor temperature (i.e. maximize catalyst longevity). Multi-objective genetic algorithm (MOGA) is chosen to optimize the input space of EO reactor MGP model to generate pareto optimal solutions which simultaneously achieve both the objectives in the best possible manner. Once the reliable models were successfully found, the models were subjected to verification and further optimization. Purpose of optimization is to find the optimum value of reactor operating parameters to achieve maximum selectivity and minimum temperature simultaneously. One of the critical tasks for optimization of any process is fixing of search space at which the optimal process conditions are to be found out. Therefore, before running the optimization a lower bound and upper bound of the process variables were fixed in consultation with plant engineers. As plant conditions are dynamic in nature, the total operational time was divided into three periods [start of run (SOR), middle of run (MOR) and end of run (EOR)] and at each running period, optimizations were performed. The lower bounds (LB) and upper bounds (UB) considered in these three cases have been depicted in Table 7.

TABLE 7
Lower bounds and upper bounds for optimization

| | x1 | x2 | x3 | x4 | x5 | x6 | x7 | x8 |
|---|---|---|---|---|---|---|---|---|
| LB | 4.98 | 17.69 | 0.21 | 21.84 | 687.78 | 161 | 275.28 | 4.98 |
| UB | 7.75 | 34.97 | 0.24 | 22.64 | 803.39 | 163 | 275.28 | 7.75 |
| LB | 4.98 | 17.69 | 0.21 | 21.84 | 687.78 | 161 | 2572.84 | 4.98 |
| UB | 7.75 | 34.97 | 0.24 | 22.64 | 803.39 | 163 | 2572.84 | 7.75 |
| LB | 4.98 | 17.69 | 0.39 | 21.84 | 687.78 | 161 | 4465.12 | 4.98 |
| UB | 7.75 | 34.97 | 0.42 | 22.64 | 803.39 | 163 | 4465.12 | 7.75 |

With the help of MOGA algorithm, a pareto diagram was developed for case 2 (MOR) (FIG. 11). All the points in the pareto curve in FIG. 11 represents the pareto non-dominated optimal solution in catalyst selectivity and reactor temperature space and all Pareto optimal solutions on the curve are considered equally good. As seen from the curve, if someone try to increase selectivity, one has to sacrifice reactor temperature. Reverse is also true, i.e., lower temperature can be achieved at the cost of lower selectivity. Other than these solutions, there is no process condition which can increase selectivity without sacrificing temperature and vice versa. Some of the pareto optimal solutions have been also tabulated in Table 7 and indicted by black circles in FIG. 9 for reference. From the output of MOGA the optimum values of input parameters can be obtained which will maximize the selectivity and minimize the temperature. All the four solutions are equally good and it is up to the plant operation engineers which criteria among selectivity or temperature they will give priority. Based on that, they can choose the optimum input parameters from the Pareto optimal solution. For example, if selectivity has more priority than reactor temperature for a particular plant, then operators should choose solution number 4 in Table 7 which will help to achieve 86.1% selectivity by sacrificing the temperature (241.53° C.). On the other hand, if the temperature has more priority than selectivity then solution number 1 in Table 7 should be chosen which will give a lower temperature by sacrificing selectivity. If temperature and selectivity have equal priority, then solution number 2 of Table 7 should be chosen.

The main advantage of such an invention is that it gives the operation engineer/DCS panel operators a strategy to run the reactor in optimum condition in real-time. In a running plant, since operators have no idea about Pareto optimal solution, operators try to optimize the plant heuristically based on their experience and knowledge. One such real-life operating point is plotted and named as ‘A’ in FIG. 11. As the operating point, A is not on the Pareto curve, it is not the optimum point and improvement scope exists both in selectivity and temperature. From this plot in FIG. 11, it can be concluded that the operator can improve the plant operating conditions in two ways: selectivity can be improved keeping the temperature same as before, or reactor temperature can be decreased keeping the catalyst selectivity as it is as shown by two arrows on FIG. 11. The only action operator has to do is to run the MOGA with proper bound in real-time, and MOGA will provide a set of optimum operating conditions that the operator needs to set in the plant. This is the potential improvement area suggested by the Pareto optimal solution.

Of course, it goes without saying that the invention is capable of outputting control data for running the reactor in optimum condition in real-time. In other words, the operation engineer/DCS panel operators do not have to input the model's output data into the control unit of the EO reactor, but the model's output data are fully automatically input into the control unit of the EO reactor. This way, the EO reactor is immediately fed with optimum control data, which prevents reading errors when interpreting the model's output data, typos when entering the data to the control unit as well as sabotage.

As a further option, an engineer can be prompted for applying the data recently output by the model. In other words, the engineer is asked whether to apply the model's output data to the control unit of the EO reactor as soon as same have been calculated. E.g. such prompt can be transferred to a personal computer or to a handheld device (smart phone, tablet, smart watch or the like) of a competent engineer via land line or wirelessly. The prompt as well as the engineers answer (accept or decline) can be transmitted via the companies Ethernet and/or the internet).

In particular, the prompt can depend on a difference between the currently applied values and the values recently output by the model. Only in case the difference exceeds a certain threshold value, a prompt and request for acceptance is sent to the engineer, whereas if not exceeded, the values recently output by the model are adopted automatically.

The threshold can be set by a user/engineer in order to match his faith in the model (e.g. based on his experience/skill level). The threshold can be defined in dependency of the current temperature of the process and/or the current selectivity of the catalyst.

TABLE 8
Pareto optimal solution

| x1 | x2 | x3 | x4 | x5 | x6 | x7 | x8 | Selectivity | Temperature |
|---|---|---|---|---|---|---|---|---|---|
| 7.75 | 18.25 | 0.31 | 22.64 | 782.15 | 161.09 | 2572.84 | 2.59 | 86.40 | 242.37 |
| 7.75 | 28.09 | 0.31 | 22.54 | 785.62 | 161.08 | 2572.84 | 2.52 | 86.71 | 243.01 |
| 7.74 | 28.18 | 0.31 | 22.17 | 785.93 | 161.09 | 2572.84 | 2.52 | 86.92 | 243.82 |
| 7.74 | 28.71 | 0.31 | 21.86 | 785.96 | 161.07 | 2572.84 | 2.53 | 87.10 | 244.53 |

### 25. Recommendation Module

Online recommendation can be generated by digital twin application as proposed by the present invention using feedforward and feedback action.

This online digital twin application provides online recommendation by using combination of feedforward and feedback action (refer FIG. 2)

### Features of Feed Forward Module

1) In feedforward module (FFM) various process parameter data are collected at fixed time intervals, e.g. every hour from plant historian.
2) Data is subjected to validation and only corrected data qualify for next step.
3) In FFM, various predictive modules are built offline from historical plant data using e.g. various AI based techniques like Genetic Programming and Partial least square (PLS). With this trained model, 24 hrs. future selectivity, temperature and EOE prediction are possible.
4) An optimization module then calculates the optimum value of controlled variables like EDC flow, ethylene & oxygen reactor inlet concentration, cycle gas pressure etc. to improve the selectivity & temperature value in future.
5) Then FFM provides online recommendation to change the control variable value within their operating bounds.
6) It also provides an estimate of how much selectivity and temperature will improve e.g. in 24 hrs future if the recommendations are followed and implemented automatically or by plant engineers in real plant.
7) In short, feedforward module, collects data from plant data historian, clean & validate the data, make predictions for reactor performance parameters (like selectivity, temperature & EOE) and recommend corrective & preventive actions which are likely to improve performance.

### Features of Feedback Module

1) The feedback module monitors the actual performance of real plant against expected performance as predicted by models in FFM.
2) It acts as a watchdog of real plant performance.
3) It may happen that the selectivity and temperature of real plant differs significantly from model predictions. Reasons are as follows but not limited to:
a) An unmeasured catalyst poison may introduce in cycle gas system through ethylene or methane. Acetylene & propane are such poisons.
b) Catalyst behavior may change during operations due to water carryover, carbonate carryover etc.
c) Selectivity & temperature may change with some unknown parameters which are either not measured or not taken into account in predictive model.
4) Feedback loop will monitor the real performance of plant (selectivity and temperature) after implementing a recommendation generated out of feedforward action.

If the improvement of selectivity and temperature is positive, then feedback action will not take any action. Program will continue and give recommendation through FFM in next cycle. But at any point in time, when the feedback action detects that performance deteriorates and not as per expected, feedback action then generates a recommendation to revert back last action and go to original position before recommendation.

This feedback recommendation will override the feedforward recommendation in current cycle. This combination of feedback & feedforward action will autocorrect any wrong move generated out of model inaccuracies or unforeseen disturbance in plant.

### 26. Online Auto Retraining Module

In reality, catalyst behavior may change with its age while running continuously inside the plant. Reasons are plenty and not fully known. Some of the reasons are (but not fully known):
i) Some of the active sites of the catalyst get damaged during operation due to poison, water or carbonate carryover, silver loss due to attrition etc.
ii) With high temperature and prolonged operation, catalyst structure and its inherent orientation may change.
iii) Some of the catalyst may change permanently due to post ignition (noticed or unnoticed).

This behavior change can be detected by slope change of selectivity and temperature curve. As catalyst behavior was changed during operation, any model built from past historical data may not be valid or model's prediction capability may reduce drastically. Hence, online retraining of model is necessary to keep the model prediction accurate all the time throughout the catalyst life.

In this application, auto retraining of model is incorporated. With this feature, the model gets retrained with e.g. last 15 days' data and always remain accurate. Even catalyst behavior is changed during operation, such change is detected by prediction error (error between selectivity (or temperature) and model provided selectivity).

As prediction error remain high for continuous about 15 days, model retrains itself automatically with recent fresh data of last 15 days. Even if such behavior change does not happen, then also the present model is retrained in every 15 days, by default.

This auto retraining facility make the model up to date all the time and improve prediction accuracy in most of catalyst change.

If this auto-retraining facility is absent from this application, the static model may become invalid, or its prediction accuracy may deteriorate significantly with time if catalyst changes its behavior.

In the following, an example is given on how auto retraining is done in a process according to the present invention:

In this application, a database is maintained for e.g. last 15 days' data. These data are collected from plant data historian system.

In each cycle, the program according to the present invention detects its own prediction accuracy, which means it calculates the error between actual selectivity seen in the plant and model predicted selectivity (same applies for temperature).

If prediction error increase significantly, or exemplary 15 days elapsed, this application runs an auto retraining of model. In this auto retraining facility, an algorithm takes the last 15 days' recent data and perform following operation to update the model coefficients. Exemplary steps performed by the auto retraining module:
i) Prediction error is calculated for each record.
ii) Overall average prediction error is calculated for the whole set of data for e.g. the last 15 days.
iii) A genetic algorithm based numerical optimization solver is run whose job is to find the updated value of model coefficients which minimizes the overall average prediction error.
iv) A lower and upper bound of model coefficient is specified (which is ±25% of present value of coefficient) and the solver finds the new optimum value within this bound while searching. This ensures the new model does not go anywhere and remain very near to present model.

FIG. 12 shows a schematic diagram of how the basic process of the present invention works: from real planned reactor I the engineer at the control center II receives sensor data and measurement results (arrow P1). According to the state of the art, the engineer takes control actions based on his knowledge about how the measured and calculated values will affect the EO production process. According to the present invention, the sensor data are also provided to a data historian III (arrow P2), where the sensor data in measurement results as well as any other valuable information about the planned performance are collected. From the data historian III, an AI based model IV can retrieve data and use training data as well as predefined references (expected results and so on) for optimizing the EO production processes parameter. Therefore, the AI based model generates a signal which either is displayed on a display of the control center II and recognized by the engineer, or immediately takes effect on the real EO reactor's control unit. Thus, the AI based model is indirectly or directly helpful when optimizing the reactor's performance (maximizing selectivity and productivity of the EO generation process).

Summarizing, and with no limiting effect in light of the enclosed set of claims, the present invention deviates from the prior art at least as follows:
1. An online real time digital twin of EO reactor is built in this disclosure which collects data from actual plant, do all internal analysis of process performance with a built in model and recommend corrective and preventive actions to plant engineers to optimize the process performance. The inventive digital twin of the EO reactor will mimic the real plant to find out the direction of future chloride change. The disclosure describes a computer model based digital twin application of EO reactor.
2. A calculation methodology is found in present invention to estimate the response time of various process parameters to affect the EO catalyst selectivity and activity.
3. A genetic programming based data driven EO reactor model (as closed form equation) is found in the present invention which can accurately and reliably predict the future selectivity and reactor temperature based on past operating parameters.
4. Closed form explainable model equations are developed for catalyst selectivity and reactor temperature, which are understandable, simple, portable, implementable in real time applications and follow the process phenomenology.
5. A calculations methodology is developed to estimate parametric co-efficient of various process parameters for selectivity and activity from plant historic actual data.
6. Since it was found that parametric co-efficient values changes along with catalyst age, a methodology was found to estimate the parametric co-efficient at different age of catalyst life.
7. An online data validation module based on detail calculations of EO reaction process was found in present invention which cross check the accuracy of measured data and rectify them if there is some measurement error.
8. A multi-objective genetic algorithm based online optimization calculations is found which optimize different process parameters of EO reactor and simultaneously maximize selectivity and activity of catalyst.
9. A recommendation procedure was found in present invention to guide the plant engineers what action they need to do to run the EO reaction process at optimum conditions all the time. A unique combination of feedforward and feedback actions are merged to generate final recommendations.
10. This ensures that EO reaction process runs at optimum conditions even if some unforeseen events happened and plant does not behave according to predictive model.
11. An online auto retraining facility is implemented in the present invention to retrain the models at regular interval of time with fresh data. This ensures the in-built model remain up-to-date always with good prediction accuracy. This also ensures the model captures and track the catalyst behavior change (if any) during its prolong run in plant.

### 27. REFERENCES

[1] S. K. Lahiri, Profit Maximization Techniques for Operating Chemical Plants.; John Wiley & Sons, 2020
[2] J. R. Koza, Genetic Programming: On the Programming of Computers by Means of Natural Selection, 1992 Cambridge. MA: The MIT Press.
[3] J. R. Koza, J. P. Rice, Genetic Programming: The Movie. 1992 Cambridge, MA: The MIT Press.
[4] M. Willis, H. Hiden, M. Hinchliffe, B. Mckay, G. W. Barton, Comput Chem Eng. 1997, 21 (SUPPL.1), S1161-S1166
[5] G. J. Gray, D. J. Murray-Smith, Y. Li, K. C. Sharman, Electronic Letters, 1996, 32, 1422-1424.
[6] B. Mckay, M. Willis, G. Barton, Comput Chem Eng., 1997, 21 (9). 981-996
[7] S. Lakshminarayanan H. Fujii, B. Grosman, E. Dassau, D. R. Lewin Computers and Chemical Engineering, 2000, 24 (2-7), 671-676
[8] B. Grossman, (2001). Nonlinear system modeling using genetic programming. MSc thesis, Technion.
[9] B. Grossman, D. R. Lewin, Computers and Chemical Engineering, 2002, 26 (4-5), 631-640.
[10] D. P. Searson, M. J. Willis, G. A. Montague, J Chemometr, 2007, 2, 592-603
[11] D. P. Searson, D. E. Leahy, M. J. Willis, (2010). GPTIPS: An open-source genetic programming toolbox for multigene symbolic regression. In Proceedings of the international multiconference of engineers and computer scientists 2010 (IMECS 2010) (pp. 77-80, Vol. 1). Hong Kong.
[12] I. Pan, D. S. Pandey, S. Das, Journal of Renewable and Sustainable Energy, 2013, 5 (6), 063129.
[13] R. Barati, R., S. A. A. S. Neyshabouri, G. Ahmadi, Powder Technology, 2014 257, 11-19.
[14] A. G. Floares, I. Luludachi, Springer Handbook of Bio-/Neuroinformatics, 2014, Springer Berlin Heidelberg, 311--326.
[15] A. H. Gandomi, A. H. Alavi, Neural Comput & Applic, 2011, 21 (1), 171-187.
[16] S. K. Lahiri, S. Chowdhury, A. Hens, K. C. Ghanta, Env. Sc & Pol Res, 2021 https://doi.org/10.1007/s11356-021-12504-w
[17] A. D. Mehr, V. Nourani, Environmental Modelling & Software, 2017 92, 239-251

1. A method of estimating a selectivity and/or activity of a catalyst in an ethylene oxide reactor comprising the steps:
Automatically acquiring process data of a reactor producing ethylene oxide by means of sensors,
Automatically determining a parametric coefficient from the acquired process data, and
Automatically calculating the selectivity and/or activity of the catalyst from the parametric coefficient.

2. The method according to claim 1, wherein the process data comprise
current sensor data, and/or
change in selectivity and/or
inlet moisture, and/or
inlet ethylene oxide and/or
inlet ethane and/or
inlet oxygen concentration and/or
inlet CO₂ concentration and/or
Cycle Gas (CG) pressure and/or
Cycle gas (CG) flow rate and/or
total chloride concentration and/or
parametric coefficients.

3. The method according to claim 1 wherein the process data of the reactor are captured by
flow sensors and/or
temperature sensors and/or
pressure sensors and/or
an online analyzer sensors (Gas chromatograph or Mass spectrometer)

4. The method according to claim 1, wherein
Determining the parametric coefficient from the acquired process data involves calculating the parametric coefficient by means of one, in particular two, AI algorithm or algorithms, respectively.

5. The method according to claim 4, wherein the AI algorithm or algorithms, respectively, comprises a combination of artificial neural network and genetic programming.

6. The method according to claim 1, wherein
Determining a parametric coefficient from the acquired process data is performed by applying the acquired process data to a genetic programming model and/or a kinetic based detail phenomenological model.

7. The method according to claim 1, wherein the Determining a parametric coefficient from the acquired process data is performed by applying the acquired process data to a combination of a genetic programming model and a second, in particular a first principle-based kinetic, model of the process.

8. The method according to claim 7, wherein the model is a first principle-based kinetic model and a prediction error of the model is minimized by an artificial intelligence-based data driven model.

9. The method according to claim 1, wherein the acquired process data comprise:
internal reactor data, in particular
an age of a catalyst, and/or
a temperature and/or
a pressure and/or
inlet moisture and/or
inlet ethylene oxide concentration or amount
an ethane concentration
and/or external reactor data, in particular
EO stripper bottom temperature and pressure and/or
Cycle water system data, in particular cycle water flow and temperature, and/or
CO2 regenerator bottom temperature and/or
CO2 removal system data, in particular carbonate flow, density, temperature.

10. The method according to claim 1 further comprising the step of
comparing the selectivity and/or activity of the catalyst to a predefined reference an
automatically generating a signal representing a from the parametric coefficient a genetic programming by using sensor data of the process for ethylene production.

11. The method according to claim 1 further comprising the step of
Validating the current chloride status of ethylene oxide reactor by a plant operation experience based heuristic rules along with genetic programming calculations.

12. The method according to claim 1 further comprising the step of
training a genetic programming by using sensor data of the process for ethylene production.

13. The method according to claim 1, wherein the process data comprise or consist of at least one of:
a) Total inlet chloride moderator concentration
b) Saturated hydrocarbon inlet concentration, in particular ethane inlet concentration
c) CO₂ inlet concentration and/or the oxygen inlet concentration, in particular both the CO₂ and oxygen inlet concentrations
d) Moisture (H₂O) inlet concentration
e) Work Rate
f) C2H4 (ethylene) inlet concentration and/or ethylene oxide inlet concentration.
g)

[symbol]

14. The method according to claim 13, wherein the process data are a combination of one or more of input variables a) to f).

15. The method according to claim 13, wherein the process data are a combination of a) and b).

16. The method according to claim 13, wherein the process data are a combination of a) to c).

17. The method according to claim 13, wherein the process data are a combination of a a) to d).

18. The method according to claim 13, wherein the process data are a combination of a) to e).
